(19) United States
(12) Patent Application Publication
(10) Pub. No.: US 2013/0165459 A1
(43) Pub. Date: Jun. 27, 2013

(54) PHARMACEUTICAL COMPOSITION AND DOSAGE FORMS OF ELINOGREL AND METHODS OF USE THEREOF

(71) Applicants: Juan Wang, Foster City, CA (US); Chandir Ramani, Brampton (CA); Joe Lambing, South San Francisco, CA (US); Christine Ye, South San Francisco, CA (US); Elizabeth Vadas, Dorval (CA); Julian Taillemite, Huningue (FR); Marie-Pierre Filliot, Geispitzen (FR); Dieter Becker, Freiburg (DE)

(72) Inventors: Juan Wang, Foster City, CA (US); Chandir Ramani, Brampton (CA); Joe Lambing, South San Francisco, CA (US); Christine Ye, South San Francisco, CA (US); Elizabeth Vadas, Dorval (CA); Julian Taillemite, Huningue (FR); Marie-Pierre Filliot, Geispitzen (FR); Dieter Becker, Freiburg (DE)

(73) Assignees: NORVARTIS PHARMA AG, Basel (CH); PORTOLA PHARMACEUTICALS, INC., San Francisco, CA (US)

(21) Appl. No.: 13/521,906

(22) PCT Filed: Jan. 12, 2011
(86) PCT No.: PCT/US11/21029

§ 371 Date: Mar. 11, 2013

Related U.S. Application Data
(60) Provisional application No. 61/294,385, filed on Jan. 12, 2010.
(60) Provisional application No. 61/329,709, filed on Apr. 30, 2010.
(60) Provisional application No. 61/334,068, filed on May 12, 2010.

(51) Int. Cl. A61K 45/06; A61K 31/64

(52) U.S. Cl. CPC A61K 45/06; A61K 31/64
USPC 514/266.24

(57) ABSTRACT

The present invention is concerned with a solid delivery form of elinogrel for the treatment of thrombosis which is notable for its multi-pH dissolution, immediate release and improved pharmacokinetic properties and stability in storage. The delivery and dosage form is a solid, oral formulation comprising: a) at least about 15% by weight elinogrel or a pharmaceutically acceptable salt thereof relative to the total weight of the overall pharmaceutical composition, and b) at least one pharmaceutically acceptable carrier. A further aspect of the present invention concerns the preparation and use of such a formulation.

### CROSS-REFERENCES TO RELATED APPLICATIONS

This application claims the benefit of U.S. Provisional Patent Application Nos. 61/294,385 (filed Jan. 12, 2010), 61/329,709 (filed Apr. 30, 2010) and 61/334,068 (filed May 12, 2010), the disclosures of which is hereby incorporated by reference in its entirety for all purposes.

### TECHNICAL FIELD OF INVENTION

The present invention relates to compositions for the delivery of pharmacologically active agents, to methods of enhancing the bioavailability of pharmacologically active agents, and to methods of treating and/or preventing disease in mammals, particularly humans, by administering a pharmacologically active agent in accordance with the invention.

### BACKGROUND OF THE INVENTION

Oral delivery of pharmacologically active agents is generally the delivery route of choice since it is convenient, relatively easy and generally painless, resulting in greater patient compliance relative to other modes of delivery. However, biological, chemical and physical barriers such as poor solubility, varying pH in the gastrointestinal tract, powerful digestive enzymes, and active agent impermeable gastrointestinal membranes, makes oral delivery of some pharmacologically active agents to mammals problematic. Compounds having the formula (I):

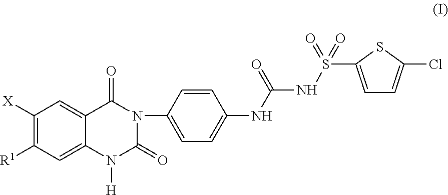

wherein: R′ is selected from the group consisting of H, halogen, —OH, —C₁₋₁₀-alkyl and C₁₋₆-alkylamino; and X is a halogen, e.g. F and I; for example, are being developed for the treatment of thrombotic complications. [4-(6-fluoro-7-methylamino-2,4-dioxo-1,4-dihydro-2H-quinazolin-3-yl)-phenyl]-5-chloro-thiophen-2-yl-sulfonylurea potassium salt (Elinogrel) has a molecular weight of 562.04 (free acid 523.95). Its pKa is about 3.3 with a logP of about 2.5 and log D (pH7.4) of about −1.6. Formulation of elinogrel has proven difficult due, at least in part, to the poor aqueous solubility of the free acid form which is <0.1 mg/ml (i.e. practically insoluble) at pH 1.0-7.4. Techniques have been disclosed for preparing sustained (or controlled) release pharmaceutical formulations of elinogrel (see e.g. U.S. patent application Ser. No. 12/618,511 filed Nov. 13, 2009. There exists a continuing need for further improvement in pharmaceutical preparations with a immediate release (IR) profile which provide still greater bioavailability of elinogrel, and other weakly acidic drugs, or their pharmaceutically acceptable salts. The present invention satisfies these and other needs.

### BRIEF SUMMARY OF THE INVENTION

Accordingly, the present invention is directed to an orally administratable pharmaceutical compositions which, unexpectedly, greatly enhances the bioavailability of a weakly acidic drug compound or a pharmaceutically acceptable salt thereof, with poor aqueous solubility, such as elinogrel. Specifically, the invention provides an orally administrable solid pharmaceutical composition comprising: a) at least about 15% by weight elinogrel or a pharmaceutically acceptable salt thereof relative to the total weight of the overall pharmaceutical composition, and b) at least one pharmaceutically acceptable carrier.

In another aspect, the invention provides an orally administrable solid pharmaceutical composition comprising: a) at least about 20% by weight elinogrel or a pharmaceutically acceptable salt thereof relative to the total weight of the overall pharmaceutical composition, and b) at least one pharmaceutically acceptable carrier. In a further aspect, the present invention provides a solid pharmaceutical composition comprising: a) about 15% to about 40% by weight elinogrel or a pharmaceutically acceptable salt thereof relative to the total weight of the overall pharmaceutical composition, and b) at least one pharmaceutically acceptable carrier. In a further aspect, the present invention provides a solid pharmaceutical composition comprising: a) about 20% to about 40% by weight elinogrel or a pharmaceutically acceptable salt thereof relative to the total weight of the overall pharmaceutical composition, and b) at least one pharmaceutically acceptable carrier.

In a further aspect, the invention is directed to a compressed solid dosage form comprising: a) solid pharmaceutical composition comprising: a) at least about 15% elinogrel or a pharmaceutically acceptable salt thereof by weight relative to the total weight of the overall pharmaceutical composition, and b) at least one pharmaceutically acceptable carrier. In another aspect, the invention is directed to a compressed solid dosage form comprising: a) solid pharmaceutical composition comprising: a) at least about 20% elinogrel or a pharmaceutically acceptable salt thereof by weight relative to the total weight of the overall pharmaceutical composition, and b) at least one pharmaceutically acceptable carrier.

In a still further aspect, the invention is directed to a method of treating or preventing a thrombotic conditions in a mammal in need thereof, which method comprises administering to said mammal an effective amount of a solid pharmaceutical composition comprising: a) at least about 15% by weight elinogrel or a pharmaceutically acceptable salt thereof relative to the total weight of the overall pharmaceutical composition, and b) at least one pharmaceutically acceptable carrier. In another aspect, the invention is directed to a method of treating or preventing a thrombotic conditions in a mammal in need thereof, which method comprises administering to said mammal an effective amount of a solid pharmaceutical composition comprising: a) at least about 20% by weight elinogrel or a pharmaceutically acceptable salt thereof relative to the total weight of the overall pharmaceutical composition, and b) at least one pharmaceutically acceptable carrier.

In another aspect the present invention provides a method to aid in dissolving elinogrel, comprising the step of: providing elinogrel in a composition with a disintegrant selected from the group consisting of croscarmellose sodium (ACDISOL®), sodium starch glycolate and crospovidone or a crystallization inhibitor selected from the group consisting of a poly(vinylpyrrolidone) and derivatives thereof, cellulosic polymers such as hydroxypropylmethylcellulose (HPMC) and hypromellose acetate succinate, xanthan gums, pectins, alginates, tragacanth and derivatives, gum arabic and derivatives, carrageenans, agar and derivatives, polysaccharides from microbiological sources, arabinogalactanes, galactomannans, dextrans, carboxylic acid and derivatives such as oleic acid, gelatin and surface active agents such as poly(vinylpyrrolidone), poloxamer, and sodium lauryl sulfate; in an amount of from at least about 3% by weight relative to the total weight of the overall pharmaceutical composition.

In a further aspect the present invention provides a method of producing a solid pharmaceutical composition comprising elinogrel; by contacting a) at least about 15% elinogrel or a pharmaceutically acceptable salt thereof by weight relative to the total weight of the overall pharmaceutical composition, with b) at least one pharmaceutically acceptable carrier. In another aspect the present invention provides a method of producing a solid pharmaceutical composition comprising elinogrel; by contacting a) at least about 20% elinogrel or a pharmaceutically acceptable salt thereof by weight relative to the total weight of the overall pharmaceutical composition, with b) at least one pharmaceutically acceptable carrier.

Another aspect of the present invention relates to a method for producing a tablet.

Further features and advantages of the invention will become apparent from the following detailed description of the invention.

### BRIEF DESCRIPTION OF THE DRAWINGS

FIG. 1A shows multi-pH dissolution profiles for Examples 5, 6, and 7. FIG. 1B shows a multi-pH dissolution profile for Examples 8, 9, 10, and 11. The five-stage dissolution method includes pH 2.0 buffer at 15, 30 min; pH 5.0 buffer at 45, 60 min; pH 6.4 buffer at 75, 90 min; pH 7.4 buffer at 105, 120 min; pH 8.2 buffer at 150 minutes of dissolution evaluation.

FIG. 2 shows the dissolution release profiles for Example 11 processed with different milling methods and screen sizes after roller compaction.

FIG. 3 shows the multi-pH (multi-stage) dissolution release profile for Example 11 compressed at different hardness.

FIG. 4 shows the single pH dissolution release profile for Example 9 on accelerated stability.

FIG. 5 shows the single pH dissolution release profile for Example 11 on accelerated stability.

FIG. 6 shows multi-pH dissolution results for IR capsules and tablets of Example 1, 5 and 11.

FIGS. 7A and 7B shows mean plasma concentration-time profiles for IR capsules and tablets of Example 1, 5 and 11 after 24 and 72 hours, respectively.

FIGS. 8A, 8B and 8C show mean plasma concentration-time profiles for individual subjects who were administered two IR capsules of Example 1, or a single IR tablet of either Example 5 or 11, respectively.

FIG. 9 show the dissolution profile of example 4, 12 and 13. Example 12 is denoted in the graph by FCT IR MgO 150 mg; and Example 13; by FCT IR 150 mg and Example 4; by capsule 75 mg.

FIG. 10 shows a dissolution profile of Examples 4, 12 and 13.

FIG. 11 shows the solubility profile of elinogrel potassium salt.

FIG. 12 shows a comparison of 75 and 150 mg film-coated tablets of elinogrel potassium salt.

### DETAILED DESCRIPTION OF THE INVENTION

### Definitions

As used herein, the following terms have the meanings ascribed to them unless specified otherwise.

The singular forms “a,” “an,” and, “the” include plural references unless the context clearly dictates otherwise. Thus, for example, reference to a compound refers to one or more compounds or at least one compound. As such, the terms “a” (or “an”), “one or more”, and “at least one” can be used interchangeably herein.

The phrase “about” as used herein is used to provide flexibility to a numerical range endpoint by providing that a given value may be “a little above” or “a little below” the endpoint accounting for variations one might see in measurements taken among different instruments, samples, and sample preparations. Typical variation among different X-ray diffractometers and sample preparations for crystalline solid forms is on the order of 0.2° 2θ. Typical variation for Raman and IR spectrometers is on the order of twice the resolution of the spectrometer. The resolution of the spectrometer used was about 2 cm⁻¹.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

The terms “therapeutic agent,” “active agent,” “active compound,” or in some cases “compound,” “bioactive agent,” “pharmaceutically active agent,” and “pharmaceutical,” and “drug” are used interchangeably herein to refer to a substance having a pharmaceutical, pharmacological, psychosomatic, or therapeutic effect. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, co-crystals, and salts and solvates of co-crystals, etc. including crystal modifications such as polymorphs and amorphous forms etc. Suitable agents for use in the present invention include, without limitation, compounds which have the formula (I):

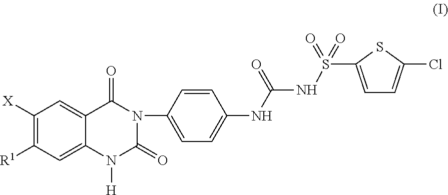

wherein:
R¹ is selected from the group consisting of H, halogen, —OH, —C₁₋₁₀-alkyl and C₁₋₆-alkylamino; and
X is a halogen, e.g. F and I, or a pharmaceutically acceptable salt thereof; and combinations thereof. In a particularly preferred embodiment, the active agent is in a salt form such as that shown below, where the symbol M represents a suitable counterion, such as potassium.

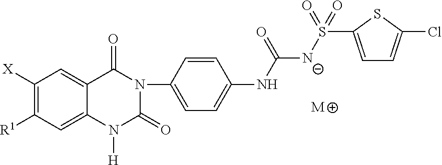

In a particularly preferred embodiment, the active agent is [4-(6-fluoro-7-methylamino-2,4-dioxo-1,4-dihydro-2H-quinazolin-3-yl)-phenyl]-5-chloro-thiophen-2-yl-sulfonylurea, in all suitable forms as disclosed for example in U.S. patent application Ser. No. 11/556,490 filed Nov. 3, 2006.

The present invention is applicable not only to [4-(6-fluoro-7-methylamino-2,4-dioxo-1,4-dihydro-2H-quinazolin-3-yl)-phenyl]-5-chloro-thiophen-2-yl-sulfonylurea, but also to other weakly acidic drugs with poor aqueous solubility.

A “polymer” as used herein refers to a composition that comprises a polymer such as cellulose derivatives, dextrans, starches, carbohydrates, base polymers, natural or hydrophilic gums, such as xanthans, alginates, gelatins; polyacrylic acids, polyvinyl alcohol (PVA), polyvinyl pyrrolidone (PVP), carbomers, combinations thereof or the like.

As used herein, the term “immediate release” as applied to drug formulations have the meanings ascribed to them in “Remington's Pharmaceutical Sciences,” 18.sup.th Ed., p. 1677, Mack Pub. Co., Easton, Pa. (1990).

Unless specified otherwise, a range of “molecular weight” of a polymer (e.g., hydroxypropyl methylcellulose (HPMC) or polyvinyl pyrrolidone (PVP) or a gelation facilitator agent (e.g., a polyethylene glycol) described below is a weighted average molecular weight (measured by gel permeation chromatography).

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

As used herein, the term “treating” refers to providing an appropriate dose of a therapeutic agent to a subject suffering from an ailment.

As used herein, the term “condition” refers to a disease state for which the compounds, compositions and methods of the present invention are being used against.

As used herein, the term “ADP-mediated disease or condition” and the like refers to a disease or condition characterized by less than or greater than normal, ADP activity. An ADP-mediated disease or condition is one in which modulation of ADP results in some effect on the underlying condition or disease (e.g., a ADP inhibitor or antagonist results in some improvement in patient well-being in at least some patients).

As used herein, “subject” refers to a mammal that may benefit from the administration of a drug composition or method of this invention. Examples of subjects include humans, and may also include other animals such as horses, pigs, cattle, dogs, cats, rabbits, rats, mice and aquatic mammals. In one specific aspect, a subject is a human.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can be determined readily by one of ordinary skill in the art. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

The term “disintegration” refers to the disintegration of tablets or capsules into their constituent granules or particles when placed in a liquid medium in the experimental condition. Complete disintegration is defined as that state in which any residue of the unit, except fragments of insoluble coating or capsule shell, remaining on the screen of the test apparatus is a soft mass having no palpably firm core. Disintegration does not imply complete solution of the unit or even of its active constituent. Suitable methods known in the art for determining the disintegration time of a solid dosage form include, e.g., the USP disintegration test <701>. The term “non-disintegrating” refers to a composition that does not fully disintegrate in an hour or less in a suitable aqueous medium determined using the USP disintegration test. The term “slow-disintegrating” refers to a composition that fully disintegrates in about an hour to about 30 minutes in a suitable aqueous medium determined using the USP disintegration test. The term “rapid-disintegrating” refers to a composition that full disintegrates in less than about 30 minutes in a suitable aqueous medium determined using the USP disintegration test.

The term “bioavailability” refers to the rate and/or extent to which a drug is absorbed or becomes available to the treatment site in the body.

As used herein, the terms “administration,” and “administering” refer to the manner in which an active agent is presented to a subject. While much of the disclosure is focused on oral administration, administration can be accomplished by other various art-known routes such as parenteral, transdermal, inhalation, implantation, ocular, otic, etc.

The term “oral administration” represents any method of administration in which an active agent can be administered through the oral route by swallowing, chewing, or sucking an oral dosage form. Such solid or liquid oral dosage forms are traditionally intended to substantially release and or deliver the active agent in the gastrointestinal tract beyond the mouth and/or buccal cavity. Examples of solid dosage forms include conventional tablets, capsules, caplets, etc.

As used herein, “oral dosage form” refers to a formulation that is prepared for administration to a subject through the oral route of administration. Examples of known oral dosage forms, include without limitation, tablets, capsules, caplets, powders, pellets, granules, solutions, suspensions, solutions and solution pre-concentrates, emulsions and emulsion pre-concentrates, etc. In some aspects, powders, pellets, granules and tablets may be coated with a suitable polymer or a conventional coating material to achieve, for example, greater stability in the gastrointestinal tract, or to achieve the desired rate of release. Moreover, capsules containing a powder, pellets or granules may be further coated. Tablets may be scored to facilitate division of dosing. Alternatively, the dosage forms of the present invention may be unit dosage forms wherein the dosage form is intended to deliver one therapeutic dose per administration. In production the drug load of the dosage forms can vary up to 2%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### DESCRIPTION OF THE EMBODIMENTS

In one embodiment, the invention provides a solid pharmaceutical composition comprising: a) at least about 15% by weight elinogrel or a pharmaceutically acceptable salt thereof relative to the total weight of the overall pharmaceutical composition, and b) at least one pharmaceutically acceptable carrier. In another embodiment, the invention provides a solid pharmaceutical composition comprising: a) at least about 20% by weight elinogrel or a pharmaceutically acceptable salt thereof relative to the total weight of the overall pharmaceutical composition, and b) at least one pharmaceutically acceptable carrier.

In another embodiment the present invention provides a solid pharmaceutical composition comprising: a) about 15% to about 40% by weight elinogrel or a pharmaceutically acceptable salt thereof relative to the total weight of the overall pharmaceutical composition, and b) at least one pharmaceutically acceptable carrier. In another embodiment the present invention provides a solid pharmaceutical composition comprising: a) about 20% to about 40% by weight elinogrel or a pharmaceutically acceptable salt thereof relative to the total weight of the overall pharmaceutical composition, and b) at least one pharmaceutically acceptable carrier.

In a further embodiment, the invention is directed to a compressed solid dosage form comprising: a) solid pharmaceutical composition comprising: a) at least about 15% elinogrel or a pharmaceutically acceptable salt thereof by weight relative to the total weight of the overall pharmaceutical composition, and b) at least one pharmaceutically acceptable carrier. In another embodiment, the invention is directed to a compressed solid dosage form comprising: a) solid pharmaceutical composition comprising: a) at least about 20% elinogrel or a pharmaceutically acceptable salt thereof by weight relative to the total weight of the overall pharmaceutical composition, and b) at least one pharmaceutically acceptable carrier.

In a still further embodiment, the invention is directed to a method of treating or preventing a thrombotic conditions in a mammal in need thereof, which method comprises administering to said mammal an effective amount of a solid pharmaceutical composition comprising: a) at least about 15% by weight elinogrel or a pharmaceutically acceptable salt thereof relative to the total weight of the overall pharmaceutical composition, and b) at least one pharmaceutically acceptable carrier. In another embodiment, the invention is directed to a method of treating or preventing a thrombotic conditions in a mammal in need thereof, which method comprises administering to said mammal an effective amount of a solid pharmaceutical composition comprising: a) at least about 20% by weight elinogrel or a pharmaceutically acceptable salt thereof relative to the total weight of the overall pharmaceutical composition, and b) at least one pharmaceutically acceptable carrier.

In another embodiment the present invention provides a method to aid in dissolving elinogrel, comprising the step of: providing elinogrel in a composition with a disintegrant selected from the group consisting of croscarmellose sodium (ACDISOL®), sodium starch glycolate and crospovidone and/or a crystallization inhibitor selected from the group consisting of a poly(vinylpyrrolidone) and a hydroxypropylmethylcellulose in an amount of from at least about 3% by weight relative to the total weight of the overall pharmaceutical composition.

In a further embodiment the present invention provides a method of producing a solid pharmaceutical composition comprising elinogrel; by contacting a) at least about 15% elinogrel or a pharmaceutically acceptable salt thereof by weight relative to the total weight of the overall pharmaceutical composition, with b) at least one pharmaceutically acceptable carrier. In another embodiment the present invention provides a method of producing a solid pharmaceutical composition comprising elinogrel; by contacting a) at least about 20% elinogrel or a pharmaceutically acceptable salt thereof by weight relative to the total weight of the overall pharmaceutical composition, with b) at least one pharmaceutically acceptable carrier.

Another embodiment of the present invention relates to a method for producing a tablet.

The composition provides a desired release profile for the active agent, specifically, in multi-stage dissolution method which mimic the gradual pH change in physiological conditions. These dosage forms gave faster and more complete release in pH 5.0-7.4 stages (after initial exposure to acid environment) compared against conventional immediate release formulations.

The active agent can be in any suitable form. In certain embodiments, the active agent can be in the form of an amorphous solid, a crystal, a granule, or a pellet. These active agent forms may facilitate certain coating processes of the active agents. Moreover, the particle can comprise a single active agent crystal (or granule or pellets or amorphous solid) or can comprise a plurality of active agent crystals (or granules or pellets or amorphous solid).

### Active Agents

In one set of embodiments, the active agents of the present invention are selected from the class of compounds in the dihydroquinazolinylphenyl thiophenyl sulfonylurea family and are useful in the treatment of conditions such as thrombosis. Illustrative examples of suitable dihydroquinazolinylphenyl thiophenyl sulfonylurea compounds for use in the present invention have the formula (I):

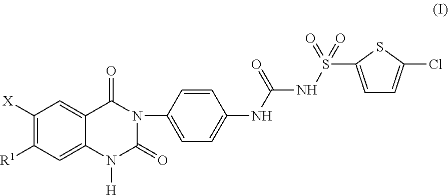

wherein:
R¹ is selected from the group consisting of H, halogen, —OH, —C₁₋₁₀-alkyl and C₁₋₆-alkylamino; and
X is a halogen, e.g. F and I.

More preferably, the agent is [4-(6-fluoro-7-methylamino-2,4-dioxo-1,4-dihydro-2H-quinazolin-3-yl)-phenyl]-5-chloro-thiophen-2-yl-sulfonylurea, in all suitable forms. In one aspect, the invention provides a solid composition, wherein the active agent is [4-(6-fluoro-7-methylamino-2,4-dioxo-1,4-dihydro-2H-quinazolin-3-ye-phenyl]-5-chloro-thiophen-2-yl-sulfonylurea potassium salt. Methods for the preparation of compounds of formula (I) are described in US-2007-0123547-A 1.

It was found that compounds of formula (I) are weak acids with poor aqueous solubility at acidic pH. Thus, in one embodiment, the active agents of the present invention are a poorly soluble weak acid compound in its salt form that has aqueous solubility of less than 0.1 mg/ml at pH 1.0-7.4 at a temperature of about 37° C. having an ionized form and an un-ionized form. The aqueous solubility increases at a higher pH (e.g. ≧1 mg/ml at pH 8 or above). In certain instances, the active agent is initially present at least partly in an ionized form. In certain other instances, the active agent is initially present in an un-ionized form. In one embodiment and as described in more detail below, the alkalizer of the compositions described herein helps to increase the solubility of the active as pH increases up to pH 10 in a hydrated media to enhance the product release profile. In another embodiment, the alkalizer of the compositions described herein helps to maintain substantially all of the active agent in its dissolved ionized form in the formulation when it is in a hydrated media.

In another set of embodiments, the active agents of the present invention are any weakly acidic drug, or a pharmaceutically acceptable salt thereof with poor aqueous solubility. As used herein, the term “active agent” includes all pharmaceutically acceptable forms of the active agent being described. For example, the active agent can be in a isomeric mixture, a solid complex bound to an ion exchange resin, or the like. In addition, the active agent can be in a solvated form. The term “active agent” is also intended to include all pharmaceutically acceptable salts, derivatives, and analogs of the active agent being described, as well as combinations thereof. For example, the pharmaceutically acceptable salts of the active agent include, without limitation, the sodium, potassium, calcium, magnesium, ammonium, tromethamine, L-lysine, L-arginine, N-ethylglucamine, N-methylglucamine and salt forms thereof, as well as combinations thereof and the like. Any form of the active agent is suitable for use in the compositions of the present invention, e.g., a pharmaceutically acceptable salt of the active agent, a free acid of the active agent, or a mixture thereof. In one embodiment, the invention provides a solid composition, wherein the active agent is [4-(6-fluoro-7-methylamino-2,4-dioxo-1,4-dihydro-2H-quinazolin-3-yl)-phenyl]-5-chloro-thiophen-2-yl-sulfonylurea potassium salt having a crystalline solid form A as described in US-2007-0123547-A1. In another embodiment, the invention provides a solid composition, wherein the active agent is [4-(6-fluoro-7-methylamino-2,4-dioxo-1,4-dihydro-2H-quinazolin-3-yl)-phenyl]-5-chloro-thiophen-2-yl-sulfonylurea potassium salt having a crystalline solid form A as described in US-2007-0123547-A1.

In some sets of embodiments, an active agent may be present in different crystal forms. The different crystalline forms of the same compound can have an impact on one or more physical properties, such as stability, solubility, melting point, bulk density, flow properties, bioavailability, etc. For example, the isolation and crystallization work indicated that there was at least four crystalline solid forms of the potassium salt of elinogrel.

The solid forms of the invention may be described by one or more of several techniques including X-ray powder diffraction, Raman spectroscopy, IR spectroscopy, and thermal methods. Further, combinations of such techniques may be used to describe the invention. For example, one or more X-ray powder diffraction patterns combined with one or more Raman spectrum may be used to describe one or more solid forms of the invention in a way that differentiates it from the other solid forms.

Although it characterizes a form, it is not necessary to rely only upon an entire diffraction pattern or spectrum to characterize a solid form. Those of ordinary skill in the pharmaceutical arts recognize that a subset of a diffraction pattern or spectrum may be used to characterize a solid form provided that subset distinguishes the solid form from the other forms being characterized. Thus, one or more X-ray powder diffraction pattern alone may be used to characterize a solid form. Likewise, one or more IR spectrum alone or Raman spectrum alone may be used to characterize a solid form. Such characterizations are done by comparing the X-ray, Raman, and IR data amongst the forms to determine characteristic peaks.

One may also combine data from other techniques in such a characterization. Thus, one may rely upon one or more x-ray powder diffraction pattern and for example, Raman or IR data, to characterize a form. For example, if one or more X-ray diffraction peak characterize a form, one could also consider Raman or IR data to characterize the form. It is sometimes helpful to consider Raman data, for example, in pharmaceutical formulations.

In yet another embodiment, an active agent is a drug that is unstable if it is in contact with simulated gastric fluid or a gel-forming matrix for a prolonged period of time at low pH (e.g., sensitive to low pH microenvironment).

In the embodiments of the invention, the active agent can be in any suitable form. For example, it can be in the form of a powder, pellet, or a granule (i.e., an aggregate of smaller units of active agent). An active agent can be pelletized or granulated using any suitable methods known in the art. Pelletization by extrusion (followed by spheronization) or granulation (wet or dry) is commonly defined as a size-enlargement process in which small particles are gathered into larger, aggregates in which the original particles can still be identified.

Any suitable granulation methods can be used to produce particles comprising an active agent. By definition, granulation is any process of size enlargement and densification whereby small particles are gathered together into larger, aggregates to render them into a free-flowing state. For example, either wet granulation or dry granulation methods can be used.

Dry granulation refers to the granulation of a formulation without the use of heat and solvent. Dry granulation technology generally includes slugging or roller compaction. Slugging consists of dry-blending, compressing the formulation into a loose, porous tablet (or slug) on a compression machine and subsequently milling it to yield the granules. Roller compaction is similar to slugging, but two counter rotating rollers are used instead of the tableting machines to form compact in the form of a ribbon for milling. See, e.g., Handbook of Pharmaceutical Granulation Technology, D. M. Parikh, eds., Marcel-Dekker, Inc. pages 102-103 (1997). Dry granulation technique is useful in certain instances, e.g., when the active agent is sensitive to heat, water or solvent.

Alternatively, the active agents are granulated with high shear mixer granulation (“HSG”) or fluid-bed granulation (“FBG”). Both of these granulation processes provide enlarged granules but differ in the apparatti used and the mechanism of the process operation. Blending and wet massing by HSG is accomplished by an impeller and a chopper in the mixer. Mixing, densification, and agglomeration of wetted materials are achieved through shearing and compaction forces exerted by the impeller. The wet mass is dried using commercial equipment such as a tray drier or a fluid-bed drier.

On the other hand, fluidization is the operation by which a mass of powder is manipulated to exhibit fluid-like characteristics using a gas or air at high velocity as the fluidization vehicle. Such a fluidized bed resembles a vigorously boiling fluid, with solid particles undergoing turbulent motion, which can be generally increased with gas velocity. FBG is then a process by which granules are produced by spraying and drying a binder solution onto a fluidized powder bed to form larger granules in a fluidbed dryer. The binder solution can be sprayed from, e.g., one or more spray guns positioned at any suitable manner (e.g., top or bottom). The spray position and the rate of spray may depend on the nature of the active agent and the binder(s) used, and are readily determinable by those skilled in the art.

Optionally, granulated active agents can be milled after wet granulation or drying. Milling can be performed using any commercially available equipment, e.g., COMIL® equipped with a screen having a suitable mesh size. The mesh size for the screen of a COMIL® can be selected depending on the size of the active agent granule or pellet desired. Typically, the mesh size can range from 0.331 inch screen (mesh 20) to 0.006 inch screen (mesh 100). The milling process aids in providing relatively uniform granule size. After the wet granulated active agents are milled, they may be further dried (e.g., in a fluidbed drier) if desired.

Typically, the mean size of the active granule can range from about 20 μm to about 3 mm, optionally about 50 μm to about 2 mm, about 100 μm to about 1 mm. Typically, the bulk density and the tap density of the active agent granules range from about 0.1 g/ml to about 1.5 g/ml, optionally about 0.3 to about 0.8 g/ml, optionally about 0.4 g/ml to about 0.6 g/ml. Bulk density is measured based on USP method (see US testing method <616>).

### Other Components and Dosage Forms

The compositions of the present invention may take the form of an immediate release tablets, pills, capsules, or the like. Preferably, the dosage form is a immediate-release tablet.

Dosage forms such as dissolving tablets, containing at least about 20% by weight elinogrel or a pharmaceutically acceptable salt and a pharmaceutically acceptable carrier and such as an alkalizer, a disintegrant, a crystallization inhibitor and combinations thereof described herein offer advantages over other traditional formulations for oral administration. For example, in multi-stage dissolution method, these dosage forms gave faster and more complete release in pH 5.0-7.4 stages (after initial exposure to acid environment) compared against conventional immediate release formulations. Similarly, the bioavailability of the therapeutic agent is increased, thereby reducing the time to onset of therapeutic activity as compared to traditional dosage forms for oral administration.

In addition, the preferred dosage forms of the present invention (e.g., at least about 20% by weight elinogrel or a pharmaceutically acceptable salt thereof and optionally containing at least one carrier such as an alkalizer, a disintegrant, a crystallization inhibitor and combinations thereof described herein) offer advantages over dosage forms using lesser amounts of active. Importantly, the larger dosage forms of the present invention help for a faster release of the active in a hydrated media. The bioavailability of the therapeutic agent is increased, and the time to onset of therapeutic activity is modulated as compared to dosage forms for oral administration that do not contain as large amount of active.

As used herein, the term “dosage form” refers to physically discrete units suitable as unitary dosages for human subjects and other mammals, each unit containing a predetermined quantity of therapeutic agent calculated to produce the desired onset, tolerability, and therapeutic effects, in association with one or more suitable pharmaceutical excipients such as carriers. Methods for preparing such dosage forms are known or will be apparent to those skilled in the art. In other embodiments, a tablet dosage form of the present invention can be prepared according to the procedures set forth, for example, in Remington: The Science and Practice of Pharmacy, 20th Ed., Lippincott, Williams & Wilkins (2003); Pharmaceutical Dosage Forms, Volume 1: Tablets, 2nd Ed., Marcel Dekker, Inc., New York, N.Y. (1989); and similar publications. The dosage form to be administered will, in any event, contain a quantity of the therapeutic agent in a therapeutically effective amount for relief of the condition being treated when administered in accordance with the teachings of this invention.

Typically, the tablet compositions of the present invention comprise at least about 20.0% by weight of the active agent (in whatever chosen form, measured as per its free acid form), and more typically from about 20.0% to about 40.0%. In some embodiments, about 35.0% by weight of the active agent is used. One skilled in the art understands that the foregoing percentages will vary depending upon the particular source of active agent utilized, the amount of active agent desired in the final formulation, as well as on the particular release rate of active agent desired.

### Carriers

The compositions of the present invention can additionally include a carrier. In one group of embodiments the composition of the present invention comprises an alkalizer, a crystallization inhibitor, a disintegrating agent, or a combination thereof.

### Alkalizers

Formulations were designed to provide an alkaline micro-environment for these compounds. The alkalizer is used to create a microenvironment in the formulation to optimize drug release after the formulation is in a hydrated media. The alkalizers of the compositions described herein are capable of raising the pH of the micro-environment for these compounds in the hydrated formulation to a pH greater than about the pKa of the active acid, irrespective of the starting pH of the media. In one embodiment, the alkalizers of the compositions described herein are capable of raising the pH of the micro-environment in the hydrated formulation to typically about 9.0-9.5, irrespective of the starting pH of media. In this way, the alkalizer helps increase the solubility of the active as pH increases up to pH 10 in a hydrated media to enhance the product release/dissolution profile from the hydrated formulation. Although pH adjusting agents may be used with the alkalizers of the present invention, one skilled in the art will appreciate that acidic agents can also be used to adjust the pH of the alkalizer as long as the alkalizer as a whole raises the pH of the micro-environment for these compounds in the hydrated formulation to greater than about the pKa of the active acid.

Suitable alkalizer agents include, but are not limited to, organic and inorganic basic compounds of a wide range of aqueous solubilities and molecular weights and the like and mixtures thereof. Representative examples of inorganic basic salts include ammonium hydroxide, alkali metal salts, alkaline earth metal salts such as magnesium oxide, magnesium hydroxide, calcium hydroxide, sodium hydroxide, potassium hydroxide, aluminum hydroxide, potassium carbonate, sodium bicarbonate and the like and mixtures thereof. In one aspect, the invention provides a solid composition wherein the alkalizer selected from the group consisting of calcium carbonate, magnesium oxide, calcium hydrogen phosphate and pharmaceutically acceptable salts thereof. The solubility and the molecular size of the alkalizer may affect its diffusion rate in the hydrated product and influence the dissolution profile of the active agent.

In one aspect, the invention provides a solid composition wherein amount of alkalizer or a mixture thereof is from at least about 1 weight percent of the total composition, more preferred from 1 to about 60 weight percent of the total composition. In one aspect, the invention provides a solid composition wherein amount of alkalizer or a mixture thereof is from at least about 3 weight percent of the total composition, more preferred from 3 to about 30 and much preferred from about 3 to about 11 weight percent of the total composition. In another aspect, the invention provides a solid composition wherein amount of alkalizer or a mixture thereof is from about 5 weight percent of the total composition, more preferred from about 5 to 15 weight percent of the total composition. In another aspect, the invention provides a solid composition wherein amount of alkalizer or a mixture thereof is from about 8 to 12 weight percent of the total composition. In one aspect, the invention provides a solid composition wherein the combined weight percent of the alkalizer is greater than or equal to the weight percent of the active. In one aspect, the invention provides a solid composition wherein the weight ratio of said alkalizer is from about 3.5 to about 10% w/w. In one aspect, the invention provides a solid composition wherein said composition comprises from about 4% w/w to about 9% w/w active; from about 4.5% w/w to about 8% w/w hydrophilic polymer; and from about 5% w/w to about 7% w/w alkalizer of the total composition.

In one aspect, the invention provides a solid composition of claim 1, wherein the composition provides at least about 20% release of the active between about 15 to about 30 minutes following administration or use.

In one embodiment, the alkalizers of the present invention are binary alkalizers, for example comprising a carbonate salt or a bicarbonate salt and a second alkalizer, for example magnesium oxide. The concentration of each alkalizer component is tailored such that the final pH of the micro-environment for these compounds in the hydrated formulation is achieved and sustained for a period of time, e.g., for at least about an hour. Selection of an appropriate weight ratio for each alkalizer component can be easily determined to achieve the dissolution profile in an aqueous environment. For example, the weight ratio of carbonate salt to bicarbonate salt can be from about 1:10 to about 10:1, preferably from about 1:5 to about 5:1, more preferably from about 1:3 to about 3:1, and still more preferably from about 1:2 to about 2:1.

Suitable carbonate salts and bicarbonate salts are described above. The amount of carbonate salt or bicarbonate salt used in the binary alkalizer is an amount that is sufficient, when used with the second alkalizer to raise pH of the micro-environment for these compounds in the hydrated formulation to a pH of about the pKa of the active acid or more, preferably about 8.5 or more, and more preferably about 9 or more (e.g., about 9-11), irrespective of the starting pH. In certain instances, the amount of the second alkalizer in the binary alkalizer is greater than or equal to the amount of the carbonate salt or bicarbonate salt. For example, the weight ratio of the second alkalizer to the carbonate salt or bicarbonate salt can be from about 1:1 to about 10:1, preferably from about 1:1 to about 5:1, and more preferably from about 1:1 to about 3:1. In certain other instances, the amount of the second alkalizer in the binary alkalizer is less than or equal to the amount of the carbonate salt or bicarbonate salt. For example, the weight ratio of the second alkalizer to the carbonate salt or bicarbonate salt can be from about 1:1 to about 1:10, preferably from about 1:1 to about 1:5, and more preferably from about 1:1 to about 1:3.

The second alkalizer is generally selected from a metal oxide such as magnesium oxide or aluminum oxide; a phosphate salt such as monobasic sodium phosphate, dibasic sodium phosphate, monobasic potassium phosphate, dibasic potassium phosphate, monobasic calcium phosphate, dibasic calcium phosphate, monobasic magnesium phosphate, dibasic magnesium phosphate, monobasic ammonium phosphate, and dibasic ammonium phosphate. However, one skilled in the art will appreciate that any metal oxide or salt of citric acid, phosphoric acid, boric acid, ascorbic acid, or acetic acid is suitable for use in the alkalizers of the present invention. The amount of the second alkalizer used in the binary alkalizer is an amount that is sufficient, when used with the carbonate salt or bicarbonate salt, to raise pH of the micro-environment for these compounds in the hydrated formulation to a pH of about the pKa of the active acid (pKa 10.3) or more. Typically this is about 9.0 to about 9.5 irrespective of the starting pH. Preferably about 8.5 or more, and more preferably about 9 or more (e.g., about 9-11), irrespective of the starting pH. In some embodiments, a metal oxide such as magnesium oxide or aluminum oxide is the preferred second alkalizer. In a particularly preferred embodiment, the metal oxide is amorphous magnesium oxide.

Alternatively, in still yet another embodiment, the alkalizers of the present invention are ternary alkalizers comprising a carbonate salt, a bicarbonate salt, and a third alkalizer. Suitable carbonate salts and bicarbonate salts are described above. The amount of carbonate salt and bicarbonate salt used in the ternary alkalizer is an amount that is sufficient, when used with the third alkalizer, to raise pH of the micro-environment for these compounds in the hydrated formulation to a pH of about the pKa of the active acid or more, preferably about 8.5 or more, and more preferably about 9 or more (e.g., about 9-11), irrespective of the starting pH.

In one group of embodiments, the alkalizer is selected from the group consisting of magnesium oxide, calcium carbonate, calcium phosphate and combinations thereof. In another group of embodiments, the alkalizer is calcium carbonate. In another group of embodiments, the alkalizer is magnesium oxide. In another group of embodiments, the alkalizer is present in an amount of about 8% to about 40% by weight. In another group of embodiments, the alkalizer is present in an amount of about 11% by weight, e.g. 11.39% by weight.

In addition other alkaline excipients can be used such as listed in Table 1.

TABLE 1
Alkaline Excipients

| | Aqueous | | | | |
|---|---|---|---|---|---|
| Alkaline | Solubility | Material | | | |
| excipient | mg/ml | state | Tm ° C. | MW | CAS No. |
| Meglumine | 1000 | solid | 128 | 195 | 6284-40-8 |
| Eglumine. | | solid | 138 | 209 | 14216-22-9 |
| Ca2CO3 | insoluble | solid | 825 | 100 | 471-34-1 |
| MgO | very slightly | solid | 2800 | 40 | 1309-48-4 |
| | soluble | | | | |
| CaHPO42H2O | insoluble | solid | | 172 | 7789-77-7 |
| Na2HPO4xH2O | very soluble | solid | | 142 | 7558-79-4 |
| NaH2PO4xH2O | 1000 | solid | | 120 | 7558-80-7 |
| KH2PO4 | 20% free sol | | | 136 | 7778-77-0 |
| K2HPO4 | >50% | | | | |
| Arginin | 150 | solid | 244 | 174 | |
| Benzathine | | | | 240 | |
| Lysine | free soluble | solid | 210 | 146 | |
| Epolamine | | | | 115 | |
| Tromethamine | 550 | solid | 171 | 121 | |
| Zn (OH)2 | | | | 99 | |

### Crystallization Inhibitors

One issue in using solubility enhancers with low solubility drugs is the crystallization of the drug in G1 fluid. Therefore, in one group of embodiments, the composition of the present invention also includes a crystallization inhibitor. As used herein, the term “crystallization inhibitor” refers to salts, ions, carbohydrates, surfactants, amino acids, polymers and other compounds which, when present in solution, decrease the crystallization of elinogrel. Crystallization inhibition of drugs can be measured with commercially available equipment e.g. CheqSol device (Sirius Analytical Instruments Ltd., Riverside, Forest Row Business Park, Forest Row, East Sussex, RH18 5DW, UK). Examples of crystallization inhibitors include, but are not limited to polyvinyl pyrrolidone, for example those products known under the registered trade marks PVP K30, PVP K29/32, in particular having a molecular weight in excess of 1 000 000, more particularly having a particle size distribution of less than 400 microns or less than 74 microns. Another example is HPMC (hydroxypropyl methyl cellulose) that are known under the trade mark Pharmacoat e.g. Pharmacoat 603.

It should be noted that crystallization inhibitors may act as stabilizing or solubilizing agents. Stabilizing agents preserve the unit activity of elinogrel in storage and may act by preventing formation of aggregates, or by preventing degradation of the elinogrel molecule (e.g. by acid catalyzed reactions). Solubilizer are mainly alkaline excipients or polymers that inhibit to a certain extend the precipitation of the drug in solution. Solubilizing agents or solubilizers increase the solubility of elinogrel above 0.1 mg/mL. Solubilizers may also raise the concentrations of elinogrel above 0.2 mg/mL and above 1 mg/mL. In one group of embodiments, compositions include the solubilizer excipient(s) or a mixture of such excipients in a ratio of 1:10 to 10:1 (drug:excipient(s)). In another group of embodiments, the crystallization inhibitor is selected from the group consisting of a poly(vinylpyrrolidone) and a hydroxypropylmethylcellulose. In another group of embodiments, the hydroxypropylmethylcellulose has a viscosity of about 5 cP. In another group of embodiments, the poly(vinylpyrrolidone) is PVP K30. In another group of embodiments, the crystallization inhibitor is present in an amount of from at least about 3% to about 50% by weight, preferably 5% to about 40% and more preferable 5% to 20% relative to the total weight of the overall pharmaceutical composition. In another group of embodiments, the crystallization inhibitor is present in an amount from at least 3% to about 11% by weight relative to the total weight of the overall pharmaceutical composition. In another group of embodiments, the crystallization inhibitor is present in an amount from at least 3% to about 6% by weight relative to the total weight of the overall pharmaceutical composition. In another group of embodiments, the crystallization inhibitor is present in an amount from about 4% by weight, e.g. about 4.22% by weight.

### Disintegrants

Where accelerated release is desired, e.g. about 20% release within a 30 minute, more particularly a 15 minute period, a disintegrant such as croscarmellose sodium (AcDiSiol), sodium starch glycolate and crospovidone, or reactive additives (effervescent mixtures) that effect rapid disintegration of the tablet in the presence of water, for example so-called effervescent tablets that contain an acid in solid form, typically citric acid, which acts in water on a base containing chemically combined carbon dioxide, for example sodium hydrogencarbonate or sodium carbonate, and releases carbon dioxide. Thus in one group of embodiments, the disintegrant is selected from the group consisting of croscarmellose, sodium starch glycolate and crospovidone. In another group of embodiments, the disintegrant is a croscarmellose. In another group of embodiments, the disintegrant is croscarmellose sodium. In another group of embodiments, the disintegrant is present in an amount from at least about 2% by weight (e.g. 2.5% by weight) relative to the total weight of the overall pharmaceutical composition. In another group of embodiments, the disintegrant is present in an amount from at least about 2% by weight (e.g. 2.5% by weight) to about 6% by weight relative to the total weight of the overall pharmaceutical composition.

### Other Carriers

As used herein, the term “carrier” also refers to a typically inert substance used as a “diluent” or vehicle for a drug such as a therapeutic agent. The term also encompasses a typically inert substance that imparts cohesive qualities to the composition. Suitable carriers for use in the compositions of the present invention include, without limitation, a binder, a gum base, and combinations thereof. Non-limiting examples of carriers and diluents include mannitol, sorbitol, xylitol, maltodextrin, lactose, dextrose, sucrose, glucose, inositol, powdered sugar, molasses, starch, cellulose, microcrystalline cellulose, polyvinylpyrrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol, and combinations thereof. These diluents can be pre-processed to improve their flowability and taste by methods known in the art such as freeze drying (see, e.g., Fundamentals of Freeze-Drying, Pharm. Biotechnol., 14:281-360 (2002); Lyophililization of Unit Dose Pharmaceutical Dosage Forms, Drug. Dev. Ind. Pharm., 29:595-602 (2003)); solid-solution preparation (see, e.g., U.S. Pat. No. 6,264,987); and lubricant dusting and wet-granulation preparation with a suitable lubricating agent (see, e.g., Remington: The Science and Practice of Pharmacy, supra). For example, Mannogem® and Sorbogem®, sold by SPI Pharma Group (New Castle, Del.), are freeze-dried processed forms of mannitol and sorbitol, respectively. Typically, the compositions of the present invention comprise from about 25% to about 90% by weight of the diluents, and preferably from about 50% to about 80%. However, one skilled in the art will appreciate that the compositions of the present invention can be made without any diluents, e.g., to produce a highly friable dosage form.

In one aspect, the invention provides a solid composition comprising a diluent selected from the group consisting of microcrystalline cellulose, lactose and mannitol.

The formulation further may comprise pH adjusting agents; antioxidants, such as butylated hydroxytoluene and butylated hydroxyanisole; plasticizers; glidants; protecting agents; elastiomeric solvents; bulking agents; wetting agents; emulsifying agents; solubilizing agents; lubricants; suspending agents; preserving agents such as methyl-, ethyl-, and propyl-hydroxy-benzoates; sweetening agents; flavoring agents; coloring agents; and disintegrating agents.

It is preferred to add such pH-adjusting acids to create and regulate a buffered microenvironment when combined with one or more alkalizers to obtain the desired delivery rate for the drug agent, Among those agents are but not limited to citric-acid, succinic acid, tartaric acid, acetic acid, and vitamin C. Preferred are buffer substances like citric acid.

The pharmaceutical formulations disclosed herein can further comprise antioxidants and chelating agents. For example, the pharmaceutical formulations can comprise butylated hydroxyanisole (BHA), butylated hydroxytoluene (BHT), propyl gallate (PG), sodium metabisulfite, ascorbyl palmitate, potassium metabisulfite, disodium EDTA (ethylenediamine tetraacetic acid; also known as disodium edentate), EDTA, tartaric acid, citric acid, citric acid monohydrate, and sodium sulfite. In one embodiment, the foregoing compounds are included in the pharmaceutical formulations in amounts in the range of about 0.01% to about 5% w/w. In one specific embodiment, the pharmaceutical formulation includes BHA, BHT, or PG used at a range of about 0.02% to about 1% and disodium EDTA, citric acid, or citric acid monohydrate used at a range of about 2% to about 5%. In a preferred embodiment, the pharmaceutical formulation includes BHA used at about 0.05% w/w.

Lubricants can be used to prevent adhesion of the dosage form to the surface of the rollers, dies and punches, and to reduce inter-particle friction. Lubricants may also facilitate ejection of the dosage form from the die cavity and improve the rate of granulation flow during processing. Examples of suitable lubricants include, without limitation, magnesium stearate, glyceryl behenate, calcium stearate, zinc stearate, stearic acid, silicon dioxide, talc, polyethylene glycol, mineral oil, carnauba wax, palmitic acid, sodium stearyl fumarate sodium laurel sulfate, glyceryl palmitostearate, myristic acid and hydrogenated vegetable oils and fats, as well as other known lubricants, and/or mixtures of two or more thereof. In one embodiment, the lubricant, if present, of the stock granulation is magnesium stearate. The compositions of the present invention can comprise from about 0% to about 10% by weight of the lubricant, and preferably from about 1% to about 5%.

In another embodiment, the composition can also optionally include an anti-adherent or glidant. Examples of glidants and/or anti-adherents suitable for use herein include but are not limited to, silicon dioxide, colloidal silicon dioxide, magnesium silicate, magnesium trisilicate, talc, and other fauns of silicon dioxide, such as aggregated silicates and hydrated silica. In another embodiment, the composition can also optionally include an opacifying agent, such as titanium dioxide, for example. In yet another embodiment, the composition can also optionally include one or more colorants, for example, iron oxide based colorant(s).

The tablet composition may further comprise a protecting agent. The protecting agent coats at least part of the therapeutic agent, typically upon the mixing of the two agents. The protecting agent may be mixed with the therapeutic agent in a ratio from about 0.1 to about 100 by weight, preferably in a ratio from about 1 to about 50, and more preferably in a ratio of about 1 to about 10. Without being bound to any particular theory, the protecting agent reduces the adhesion between the therapeutic agent and the binder so that the therapeutic agent may be more easily released from the binder. In this way, the therapeutic agent may be delivered in the stomach within about 7 to about 12 hours, preferably within about 12 hours. Materials suitable as protecting agents are discussed in detail above and may be used alone or in combination in the tablet compositions of the present invention.

The tablet composition may also comprise one or more elastomeric solvents such as rosins and resins. Non-limiting examples of such solvents are discussed in detail above and may be used alone or in combination in the tablet compositions of the present invention. In addition, the tablet composition may further comprise waxes such as beeswax and microcrystalline wax, fats or oils such as soybean and cottonseed oil, and combinations thereof. Moreover, the tablet composition may additionally include plasticizers such as softeners or emulsifiers. Such plasticizers may, for example, help reduce the viscosity of the gastric solution of the dissolved tablet to a desirable consistency and improve its overall texture and bite and help facilitate the release of the therapeutic agent. Non-limiting examples of such plasticizers are discussed in detail above and may be used alone or in combination in the tablet compositions of the present invention.

In one embodiment of the stock granulation, the bulking agent is microcrystalline cellulose and/or lactose monohydrate, the binder, if present, is pregelatinized starch, the disintegrant, if present, is sodium starch glycolate, croscarmellose sodium, crospovidone, or combinations thereof; the lubricant, if present, is magnesium stearate and the glidant and/or anti-adherent, if present, is colloidal silicon dioxide and/or talc.

Sweetening agents can be used to improve the palatability of the composition by masking any unpleasant tastes it may have. Examples of suitable natural or artificial sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Hydrogenated starch hydrolysate, and the potassium, calcium, and sodium salts of 3,6-dihydro-6-methyl-1-1,2,3-oxathiazin-4-one-2,2-dioxide may also be used. The compositions of the present invention can comprise from about 0% to about 80% by weight of the sweetening agent, preferably from about 0.5% to about 75%, and more preferably from about 0.5% to about 50%.

Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like. Coloring agents can be used to color code the composition, for example, to indicate the type and dosage of the therapeutic agent therein. Suitable coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present invention can comprise from about 0% to about 10% by weight of the flavoring and/or coloring agent, preferably from about 0.1% to about 5%, and more preferably from about 2% to about 3%.

Preparation of Solid Compositions Comprising Active Agent into Tablets

Any suitable methods can be used to mix the formulation comprising the active agent. In one embodiment, the active agent and carrier are combined, mixed and the mixture may be directly compressed into a tablet. Typically, one or more vehicles or additives may be added to the mixture to improve flow and compressible characteristics. These additives include, for example, lubricants, such as magnesium stearate, zinc stearate, stearic acid, talc, and the like; flavors; and sweeteners. Direct compression has advantages, such as reducing cost, time, operational pace, and machinery; preventing active agent-excipient interaction; and less instability of active agent. Direct blending or dry granulation can also eliminate the possible pollution by organic solvent.

In another embodiment, some of the formulation components may be partially granulated prior to compression or all of the formulation components may be granulated prior to compression. For example, the active agent, alone can also be granulated prior to mixing.

Any suitable granulation methods can be used to mix the formulation. In one embodiment, a wet granulation process can be used to mix one or more components of the formulation. For example, high shear granulation or fluid-bed granulation processes can be used. Any suitable commercially available granulation equipment can be used in these processes.

After the granulation of one or more components of the formulation, optionally, granulated formulation can be milled. Milling can be performed using any suitable commercially available apparatus, e.g., COMIL® equipped with a screen of a suitable mesh size. The mesh size for the screen of a COMIL® can be selected depending on the size of the granules desired. After wet granulated active agents are milled, they may be further dried (e.g., in a fluid-bed) if desired.

After preparing the formulation as described above, the formulation is compressed into a tablet form. This tablet shaping can be done by any suitable means, with or without compressive force. For example, compression of the formulation after the granulation step or blending can be accomplished using any tablet press, provided that the tablet composition is adequately lubricated unless an external lubrication process is used. The level of lubricant in the formulation is typically in the range of 0.5-2.0%, e.g. with magnesium stearate which is most commonly used as a lubricant. Many alternative means to effectuate this step are available, and the invention is not limited by the use of any particular equipment. The compression step can be carried out using a rotary type tablet press. The rotary type tableting machine has a rotary turret with multiple stations of dies and punches. The formulation is fed into the die and is subsequently compressed.

The tablet compositions can have any desired shape, size, and texture. The diameter and shape of the tablet depends on the molds, dies, and punches selected for the shaping or compression of the granulation composition. For example, tablets can be discoid, oval, oblong, round, cylindrical, triangular, and can have the shape of a stick, tab, pellet, sphere, and the like. Similarly, the tablet can be any desirable color. For example, the tablet can be any shade of red, blue, green, orange, yellow, violet, indigo, and mixtures thereof, and can be color coded to indicate the type and dosage of the therapeutic agent therein. The tablets may be scored to facilitate breaking. The top or lower surface can be embossed or debossed with a symbol or letters. The tablets can be individually wrapped or grouped together in pieces for packaging by methods well known in the art.

The compression force can be selected based on the type/model of press, what physical properties are desired for the tablets product (e.g., desired, hardness, friability, etc.), the desired tablet appearance and size, and the like. Typically, the compression force applied is such that the compressed tablets have a hardness of at least about 2 kP. These tablets generally provide sufficient hardness and strength to be packaged, shipped or handled by the user. If desired, a higher compression force can be applied to the tablet to increase the tablet hardness. However, the compression force is preferably selected so that it does not cause capping or lamination of tablets. Preferably, the compression force applied is such that the compressed tablet has a hardness of less than about 20 kP.

Typically, the final tablet will have a weight of about 50 mg to about 2000 mg, more typically about 100 mg to about 1000 mg, or about 150 mg to about 500 mg, or about 300 mg to about 450 mg. In one aspect, the invention provides a solid composition wherein the amount of active agent is about 450 mg. In one aspect, the invention provides a solid composition wherein the amount of active agent is about 300 mg. In one aspect, the invention provides a solid composition wherein the amount of active agent is about 250 mg. In one aspect, the invention provides a solid composition wherein the amount of active agent is about 225 mg. In one aspect, the invention provides a solid composition wherein the amount of active agent is about 200 mg. In one aspect, the invention provides a solid composition wherein the amount of active agent is about 175 mg. In one aspect, the invention provides a solid composition wherein the amount of active agent is about 150 mg. In one aspect, the invention provides a solid composition wherein the amount of active agent is about 125 mg. In another aspect, the invention provides a solid composition wherein the amount of active agent is about 100 mg. In one aspect, the invention provides a solid composition wherein the amount of active agent is about 75 mg. In one aspect, the invention provides a solid composition wherein the amount of active agent is about 60 mg. In one aspect, the invention provides a solid composition wherein the amount of active agent is about 50 mg. In one aspect, the invention provides a solid composition wherein the amount of active agent is about 40 mg. In one aspect, the invention provides a solid composition wherein the amount of active agent is about 30 mg. In another aspect the invention provides a solid compositions wherein the amount of active agent is from about 30 to about 300 mg; preferably, from about 30 mg to about 150 mg.

If desired, other modifications can be incorporated into embodiments of the invention. For example, modification of drug release through the tablet matrix of the present invention can also be achieved by any known technique, such as, e.g., application of various coatings, e.g., ion exchange complexes with, e.g., Amberlite IRP-69.

Capsules may be prepared by filling the blend along with suitable excipients into gelatin capsules, using a suitable filling machine.

The pharmaceutical formulations of the invention can be packaged in any packaging that facilitates stability of the drug formulation. For example, sealed high density polyethylene (HDPE) bottles containing silica gel desiccant or aluminum blister lined with PVC (thermoform PVC blister) or aluminum—aluminum blister can be used. Use of such packaging helps to control unwanted oxidation and moisture ingress of the product.

Preferably, the ingredients in the pharmaceutical compositions of the instant invention are homogeneously or uniformly mixed throughout the solid dosage form. Whether the active agents are distributed randomly or non-randomly, a tablet can comprise one or more types of active agent, and/or one or more types of coating materials. The non-random distribution of active agent can be represented quantitatively by different amounts in different layers or qualitatively by having different forms of active agent in different layers, e.g., as having more coating materials in the particle in the outer layers as compared to the inner layers of the tablet, or, vice versa.

### Methods of Administration

Methods for preventing or treating thrombosis in a mammal embraced by the invention administering a pharmaceutical composition of the invention as described above. Pharmaceutical compositions of the invention are suitable for use alone or as part of a multi-component treatment regimen for the prevention or treatment of cardiovascular diseases, particularly those related to thrombosis. For example, a compound or pharmaceutical composition of the invention may be used as a drug or therapeutic agent for any thrombosis, particularly a platelet-dependent thrombotic indication, including, but not limited to, chronic coronary heart disease (cCHD), Acute coronary syndrome (ACS), acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura, thrombotic and restenotic complications following invasive procedures, e.g., angioplasty, carotid endarterectomy, post CABG (coronary artery bypass graft) surgery, vascular graft surgery, stent placements and insertion of endovascular devices and protheses, and hypercoagulable states related to genetic predisposition or cancers. In other groups of embodiments, the indication is selected from the group consisting of percutaneous coronary intervention (PCI) including angioplasty and/or stent, acute myocardial infarction (AMI), unstable angina (USA), coronary artery disease (CAD), transient ischemic attacks (TIA), stroke, peripheral vascular disease (PVD), Surgeries-coronary bypass, carotid endarectomy. In another group of embodiments, the indication is coronary heart disease or acute coronary syndrome.

Pharmaceutical compositions of the invention may also be used as part of a multi-component treatment regimen in combination with other therapeutic or diagnostic agents in the prevention or treatment of thrombosis in a mammal. In certain preferred embodiments, compounds or pharmaceutical compositions of the invention may be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, enoxaparin, glycoprotein (GP) 2b/3a inhibitors, aspirin, statins, angiotensin-converting enzyme (ACE) inhibitors or warfarin or anti-inflammatories (non-steriodal anti-inflammatories, cyclooxygenase II inhibitors); and Elinogrel administered by other means. Co-administration may also allow for application of reduced doses of both the anti-platelet and the thrombolytic agents and therefore minimize potential hemorrhagic side-effects. Compounds and pharmaceutical compositions of the invention may also act in a synergistic fashion to prevent reocclusion following a successful thrombolytic therapy and/or reduce the time to reperfusion.

The compositions of the invention may be administered orally in an effective amount within the dosage ranges described herein in a regimen of single or multiple (twice, etc.) daily or single or multiple weekly doses.

Importantly, the compositions of the present invention provide the rapid and predictable delivery of a active agent with surprisingly low inter-subject variability in terms of maximum plasma concentration (Cₘₐₓ) and the time to reach the maximum plasma concentration (Tₘₐₓ) by modulating the pH around the active. In particular, the delivery of the therapeutic agent optimizes absorption. As a result, the therapeutic agent can reach the systemic circulation in a substantially shorter period of time and at a substantially higher concentration than with traditional oral (e.g., tablet) administration.

In addition, the compositions of the present invention offer advantages over compositions for oral administration that do not contain a carrier (e.g. alkalizer, disintegrant, crystallization inhibitor or combination thereof) described herein. In particular, because the alkalizer, disintegrant, crystallization inhibitor or combination thereof in the compositions of the present invention can help increase the solubility of the active as pH increases up to pH 10 and/or prevent crystallization in a hydrated media to enhance the product release profile, the therapeutic agent reaches the systemic circulation in a substantially shorter period of time (e.g., reducing the time to onset of therapeutic activity) and at a substantially higher concentration than with compositions for oral administration that do not contain the carrier.

The compositions of the present invention have particular utility in the area of human and veterinary therapeutics. The compositions of the present invention may be administered to deliver an active agent to any animal in need thereof, including, but not limited to, mammals, such as rodents, cows, pigs, dogs, cats, and primates, particularly humans. Generally, administered dosages will be effective to deliver picomolar to micromolar concentrations of the active agent to the appropriate site. Administration of the compositions of the present invention is preferably carried out via any of the accepted modes of oral administration.

The following examples are intended for illustration only, are not intended to limit the scope of the invention. The contents of all U.S. patents and other references cited in this application are hereby incorporated by reference in the entirety.

### EXAMPLES

### General

Commonly used pharmaceutical excipients were used in the general formulations including: microcrystalline cellulose, Lactose Fastflo were used alone or in combination as a diluent in the formulations. Talc was used as a glidant and magnesium stearate used as a Lubricant in the formulations. Water and non-water soluble solubility enhancers including PVP and HPMC were also used. In addition suitable disintegrant, e.g. Ac-Di-Sol® or crospovidone (PVPP), were used to ensure rapid disintegration.

A wet granulation process was not used to make Compound 1 formulations as Compound 1 is moisture sensitive.

The packaging format used for packaging the core tablets for both formulations were 75 cc round white HDPE bottles with desiccant 2 gm canister and child resistant closure with induction seal. Several different formulations were made for a 50-150 mg Immediate Release (IR) capsule or tablet with a weight of up to about 450 to about 650 mg. The dosing strengths refer to the free acid quantity of Compound 1, potassium salt. The details of the formulations are summarized in the following Tables.

### Example 1

### 50 mg Elinogrel Immediate Release Capsule (Control Formulation)

| Ingredient | Function | % w/w |
|---|---|---|
| Elinogrel (potassium salt) (Form B) | Active | 12.5 |
| Mannitol | Filler | 84.5 |
| Talc | Glidant | 2.0 |
| Magnesium stearate | Lubricant | 1.0 |

### Preparation

The composition (Form B) is prepared by weighing and mixing elinogrel and excipients except magnesium stearate. The mixture is sieved and mixed and sieved magnesium stearate is added. The composition is blended, dry granulated by roller compaction and milling and then filled into capsules.

### Example 2

### 75 mg Elinogrel Immediate Release Hydroxypropylmethyl Cellulose (HPMC) Capsule (Control Formulation)

| Ingredient | Function | % w/w |
|---|---|---|
| Elinogrel (potassium salt) (Form A) | Active | 21.41 |
| Mannitol DC | Filler | 75.64 |
| Talc PH | Glidant | 1.97 |
| Magnesium stearate (external phase) | Lubricant | 0.98 |

### Preparation

The composition is prepared by weighing and mixing elinogrel (Form B) and excipients except magnesium stearate. The mixture is sieved and mixed and sieved magnesium stearate is added. The mixture is blended and dry granulated by roll compaction and milling and then filled into capsules.

### Example 3

### 75 mg Elinogrel Immediate Release Hydroxypropylmethyl Cellulose (HPMC) Capsule (Control Formulation)

| Ingredient | Function | % w/w |
|---|---|---|
| Elinogrel (potassium salt) (Form A) | Active | 21.63 |
| Mannitol DC | Filler | 75.42 |
| Talc PH | Glidant | 1.97 |
| Magnesium stearate (external phase) | Lubricant | 0.98 |

### Preparation

The composition is prepared by weighing and mixing elinogrel (Form A) and excipients except magnesium stearate. The mixture is sieved and mixed and sieved magnesium stearate is added. The mixture is blended and dry granulated by roll compaction and milling and then filled into HPMC capsules.

### Example 4

### 150 mg Elinogrel Capsules (Control Formulation)

| Ingredient | Function | % w/w |
|---|---|---|
| Elinogrel (potassium salt | Active | 21.41 |
| polymorph A) (unmilled) (Form A) | | |
| Mannitol direct compression | Filler | 75.64 |
| Talc | Glidant | 1.97 |
| Magnesium Stearate (External | Lubricant | 0.98 |
| phase) | | |
| HPMC capsule size 0 | | 95 mg |
| HPMC capsule content weight | | 406 mg |

### Preparation

The composition is prepared by weighing and mixing elinogrel (Form A) and excipients except magnesium stearate. The mixture is sieved and mixed and sieved magnesium stearate is added. The mixture is blended and dry granulated by roll compaction and milling and then filled into capsules.

### Example 5

### 100 mg Elinogrel Immediate Release Tablet

| Ingredient | Function | % w/w |
|---|---|---|
| Elinogrel (potassium salt) | Active | 22.22 |
| Lactose monohydrate (Fast Flo) | Filler | 20.03 |
| Microcrystalline cellulose | Filler | 22.75 |
| Calcium carbonate (90S ultra) | Alkalizer | 20.0 |
| Magnesium Oxide (Heavy) | Alkalizer | 8.0 |
| Croscarmellose Sodium | Disintegrant | 5.0 |
| Talc | Glidant | 1.0 |
| Magnesium Stearate (Non-Bovine) | Lubricant | 0.75 |
| Colloidal Silicon Dioxide | Glidant | 0.25 |

One formulation approach investigated the use of alkalizers such as Calcium Carbonate and Magnesium Oxide Heavy in combination with disintegrant (ACDISOL®) to create an alkalizing environment for the drug to quickly disperse and/or dissolve in gastric pH as the drug is insoluble up to pH 6.8. Avicel PH 102 and Lactose Fast Flo were used in combination as diluents. Talc and Colloidal Silicon Dioxide was used as a glidant and Magnesium Stearate used as a lubricant in the formulations.

### Preparation

Initial preparation of elinogrel immediate release tablets involves dry blending and compression. Elinogrel immediate release tablets 100 mg were manufactured using either a direct blend or roller compaction process. The batch size was approximately 200 g to 1 kg. The dosing strength refers to the free base quantity of elinogrel, potassium salt.

The direct compression process involved dry blending the API with flowable excipients, alkalizers, disintegrant, glidant and lubricant followed by compression. The diluents were mixed for two minutes (Blend 1) in a 1 qt V-shell. The drug substance was separately mixed in a 1 qt v-shell blender for 2 mins with the alkalizer(s), as the drug is soluble in a basic environment (Blend 2). The drug-alkalizer and the diluent blends were mixed together along with disintegrant for 6 mins. The final blend was mixed with glidant and lubricant for 1 min and 2 mins, respectively. The lubricated blend was compressed using a Piccola single layer press fitted with 7/16″ plain round concave tooling at a required target weight of 450 mg.

The roller compaction process involved dry blending the API with flowable excipients, alkalizer, disintegrant, glidant followed by roller compaction. Blend was then lubricated prior to compression into tablets. The diluents were mixed for 2 mins (Blend 1) in a lqt V-shell. The drug substance was separately mixed in 1 qt v-shell blender for 2 mins with the alkalizer(s), as the drug is soluble in a basic environment (Blend 2). The drug-alkalizer and the diluent blends were mixed together along with disintegrant (½ quantity) and mixed for 6 mins. The blend was mixed with glidant and lubricant (½ quantity) for 1 min and 2 mins, respectively. The blend was discharged and compacted using a Vector roller compactor TFC220. The roller compacted granules were further lubricated with the remaining half quantity of ACDISOL° and Magnesium Stearate for 2 mins in the V-shell. The final lubricated blend was then compressed using a Piccola single layer press fitted with 7/16″ plain round concave tooling at a required target tablet weight of 450 mg.

### Example 6

### 100 mg Elinogrel Immediate Release Tablet

Another formulation approach investigated use of alkalizers such as Calcium Carbonate, Magnesium Oxide Heavy in combination with disintegrant (ACDISOL®) and HPMC 5 cps were utilized to create an alkalizing environment for the drug to quickly disperse and/or dissolve in gastric pH and HPMC 5 cps was added to inhibit crystallization of elinogrel in acidic pH. Avicel PH 102 and Lactose Fast Flo were used in combination as diluents. Talc and Colloidal Silicon Dioxide was used as a glidant and Magnesium Stearate used as a lubricant in the formulations.

| Ingredient | Function | % w/w |
|---|---|---|
| Elinogrel (potassium salt) (Form B) | Active | 22.22 |
| Lactose monohydrate | Filler | 20.03 |
| Microcrystalline cellulose | Filler | 20.0 |
| Calcium carbonate | Alkalizer | 20.0 |
| Magnesium Oxide | Alkalizer | 8.0 |
| Croscarmellose Sodium | Disintegrant | 5.0 |
| HPMC 5 cps | Polymer | 3.0 |
| Talc | Glidant | 1.0 |
| Magnesium Stearate | Lubricant | 0.75 |

### Example 7

### 100 mg Elinogrel Immediate Release Tablet

Another formulation approach investigated use of alkalizers such as Calcium Carbonate, Magnesium Oxide Heavy in combination with disintegrant (ACDISOL®) and PVP K29/32 to create an alkalizing environment for the drug to quickly disperse and/or dissolve in gastric pH and PVP K29/32 was added to inhibit crystallization of elinogrel in acidic pH as well as solubilizing elinogrel in aqueous media. Avicel PH 102 and Lactose Fast Flo were used in combination as diluents. Talc and Colloidal Silicon Dioxide was used as a glidant and Magnesium Stearate used as a lubricant in the formulations

| Ingredient | Function | % w/w |
|---|---|---|
| Elinogrel (potassium salt) (Form B) | Active | 22.22 |
| Lactose monohydrate (Fast Flo) | Filler | 20.03 |
| Microcrystalline cellulose (Avicel | Filler | 20.0 |
| PH 102 | | |
| Calcium carbonate (90S ultra) | Alkalizer | 20.0 |
| Magnesium Oxide (Heavy) | Alkalizer | 8.0 |
| ACDISOL ® | Disintegrant | 5.0 |
| PVP K29/32 | Crystallization inhibitor | 3.0 |
| Talc | Glidant | 1.0 |
| Magnesium Stearate (Non-Bovine) | Lubricant | 0.75 |

### Example 8

### 100 mg Elinogrel Immediate Release Tablet

Another formulation approach investigated use of alkalizers such as Calcium Carbonate, Magnesium Oxide Heavy in combination with super disintegrant (ACDISOL®) and HPMC 5 cps were utilized to create an alkalizing environment for the drug to quickly disperse and/or dissolve in gastric pH and HPMC 5 cps was added to inhibit crystallization of elinogrel in acidic pH.

| Ingredient | Function | % w/w |
|---|---|---|
| Elinogrel (potassium salt) (Form B) | Active | 22.22 |
| Lactose monohydrate | Filler | 21.03 |
| Microcrystalline cellulose | Filler | 21.0 |
| Calcium carbonate | Alkalizer | 20.0 |
| Magnesium Oxide | Alkalizer | 8.0 |
| ACDISOL ® | Disintegrant | 3.0 |
| HPMC 5 cps | Crystallization inhibitor | 3.0 |
| Talc | Glidant | 1.0 |
| Magnesium Stearate | Lubricant | 0.75 |

### Example 9

### 100 mg Elinogrel Immediate Release Tablet

Another formulation approach investigated use of alkalizers such as Calcium Carbonate, Magnesium Oxide Heavy in combination with disintegrant (ACDISOL®) and HPMC 5 Cps were utilized to create an alkalizing environment for the drug to quickly disperse and/or dissolve in gastric pH and HPMC 5 cps was added to inhibit crystallization of elinogrel in acidic pH.

| Ingredient | Function | % w/w |
|---|---|---|
| Elinogrel (potassium salt) (Form B) | Active | 22.22 |
| Lactose monohydrate | Filler | 21.03 |
| Microcrystalline cellulose | Filler | 20.0 |
| Calcium carbonate | Alkalizer | 20.0 |
| Magnesium Oxide | Alkalizer | 8.0 |
| ACDISOL ® | Disintegrant | 3.0 |
| HPMC 5 cps | Crystallization inhibitor | 5.0 |
| Talc | Glidant | 1.0 |
| Magnesium Stearate | Lubricant | 0.75 |

### Example 10

### 100 mg Elinogrel Immediate Release Tablet

Another formulation approach investigated use of alkalizers such as Calcium Carbonate, Magnesium Oxide Heavy in combination with disintegrant (ACDISOL®) and PVP K29/32 to create an alkalizing environment for the drug to quickly disperse and/or dissolve in gastric pH and PVP K29/32 was added to inhibit crystallization of elinogrel in acidic pH.

| Ingredient | Function | % w/w |
|---|---|---|
| Elinogrel (potassium salt) (Form B) | Active | 22.22 |
| Lactose monohydrate (Fast Flo) | Filler | 21.03 |
| Microcrystalline cellulose (Avicel | Filler | 21.0 |
| PH 102) | | |
| Calcium carbonate (90S ultra) | Alkalizer | 20.0 |
| Magnesium Oxide (Heavy | Alkalizer | 8.0 |
| ACDISOL ® | Disintegrant | 3.0 |
| PVP K29/32 | Crystallization inhibitor | 3.0 |
| Talc | Glidant | 1.0 |
| Magnesium Stearate | Lubricant | 0.75 |

### Example 11

### 100 mg Elinogrel Immediate Release Tablet

Another formulation approach investigated use of alkalizers such as Calcium Carbonate, Magnesium Oxide Heavy in combination with disintegrant (ACDISOL®) and PVP K29/32 to create an alkalizing environment for the drug to quickly disperse and/or dissolve in gastric pH and PVP K29/32 was added to inhibit crystallization of elinogrel in acidic pH

| Ingredient | Function | % w/w |
|---|---|---|
| Elinogrel (potassium salt) (Form B) | Active | 22.22 |
| Lactose monohydrate (Fast Flo) | Filler | 20.03 |
| Microcrystalline cellulose (Avicel | Filler | 20.0 |
| PH 102) | | |
| Calcium carbonate (90S ultra) | Alkalizer | 20.0 |
| Magnesium Oxide (Heavy | Alkalizer | 8.0 |
| ACDISOL ® | Disintegrant | 3.0 |
| PVP K29/32 | Crystallization inhibitor | 5.0 |
| Talc | Glidant | 1.0 |
| Magnesium Stearate | Lubricant | 0.75 |

### FCT—Film Coated Tablet

### Example 12

### 150 mg Elinogrel Film Coated Tablet

| Ingredient | Function | % w/w |
|---|---|---|
| Elinogrel (potassium salt, Form A) | Active | 37.05 |
| PVP K30 | Crystallization | 4.22 |
| | inhibitor | |
| Magnesium oxide | Alkalizer | 11.39 |
| Destab Calcium Carbonate 90S ultra | Alkalizer | 28.48 |
| PVPP XL | Disintegrant | 5.49 |
| Cellulose MK GR | Filler | 9.14 |
| Aerosil 200 PH | Glidant | 0.43 |
| Magnesium Stearate (External phase) | Lubricant | 0.85 |
| Tablet core weight | | 460 mg |

HPMC Coating (Basic lacquer composition form Colorcon Ldt, UK, known as Opadry):

| Ingredient | % w/w |
|---|---|
| Basic coat White | 0.53 |
| Basic coat Yellow | 1.74 |
| Basic coat Red | 0.53 |
| Basic coat Black | 0.15 |
| Coat weight | 14 mg |

### Preparation

The composition (Form B) is prepared by weighing and mixing elinogrel and excipients except magnesium stearate. The mixture is sieved and mixed and sieved magnesium stearate is added. The composition is blended and formed into tablets by direct compression. The tablets are dedusted and subsequently coated with HPMC containing lacquer in a coating pan. The batch size is about 15 kg. The in-process controls are as follows (target values):

| Control | Range | unit |
|---|---|---|
| Tablet size | 15 * 5.9 | mm |
| Tablet thickness | 4.9-5.3 | mm |
| Crushing strength | >100 | N |
| Fiability (500 rpm) | <0.8 | % |
| Disintegration time | <10 | Min |

### Example 13

### 150 mg Elinogrel Film Coated Tablet

| Ingredient | Function | % w/w |
|---|---|---|
| Elinogrel (potassium salt, Form A) | Active | 36.68 |
| PVP K30 | Crystallization inhibitor | 4.22 |
| Calcium Hydrogen Phosphate 2AQ | Filler | 21.10 |
| Lactose spray dried | Filler | 14.35 |
| PVPP XL | Disintegrant | 4.24 |
| Cellulose (Avicel PH102) | Filler | 15.07 |
| Aerosil 200 PH | Glidant | 0.46 |
| Magnesium Stearate (External | Lubricant | 0.93 |
| phase) | | |
| Tablet core weight | | 460 mg |

HPMC Coating (Basic lacquer composition form Colorcon Ldt, UK, known as Opadry):

| Ingredient | % w/w |
|---|---|
| Basic coat White | 0.53 |
| Basic coat Yellow | 1.74 |
| Basic coat Red | 0.53 |
| Basic coat Black | 0.15 |
| Film coat weight | 14 mg |

### Preparation

The composition is prepared by weighing and mixing elinogrel (Form A) and excipients except magnesium stearate. The mixture is sieved and mixed and sieved magnesium stearate is added. The composition is blended and formed into tablets by direct compression. The tablets are dedusted and subsequently coated with HPMC containing lacquer in a coating pan. The batch size is about 15 kg. The in-process controls are as follows (target values):

| Control | Range | unit |
|---|---|---|
| Tablet size | 15 * 5.9 | mm |
| Tablet thickness | 4.9-5.3 | mm |
| Crushing strength | >100 | N |
| Fiability (500 rpm) | <0.8 | % |
| Disintegration time | <10 | Min |

### Example 14

### Testing of Example 4, 12 and 13

| Control | Example 12 | 13 | 4 |
|---|---|---|---|
| Dosage form | Film-coated | Film-coated | Capsule |
| | tablet | tablet | |
| Capsule/Tablet size mm | 15 * 5.9 | 15 * 5.9 | Size 0 |
| Tablet thickness mm | 5.0 | 5.1 | na |
| Crushing strength N | 165 | 200 | na |
| Fiability (500 rotations) % | 0.2 | 0.1 | na |
| Disintegration time Min | 1.1 | 2.2 | 5.5 |

### Dissolution Profile

### Dissolution Method: Basket 100 rpm Na Phosphate Buffer pH7.4 900 ml

| | Example no. | | | |
|---|---|---|---|---|
| | 4 | 13 | 12 | |
| Time | Capsule | FCT IR | FCT IR + MgO | |
| min | 75 mg | 150 mg | 150 mg | |
| 0 | 0 | 0 | 0 | |
| 15 | 92 | 71 | 75 | |
| 30 | 96 | 75 | 90 | |
| 45 | 97 | 77 | 95 | |
| 60 | 97 | 77 | 96 | |
| 75 | 98 | 90 | 98 | infinity test |
| | | | | 200 rpm |

Dissolution Method: Paddle 50 rpm, 5-pH Stages (Target pH 2.0, 5.0, 6.4, 7.4, and 8.0)

| | Example no. | | |
|---|---|---|---|
| | 4 | 13 | 12 |
| | Capsule | FCT IR | FCT IR + MgO |
| Time min | 75 mg | 150 mg | 150 mg |
| 0 | 0 | 0 | 0 |
| 30′ pH2 | 0. | 1 | 1 |
| 45′ pH5 | 28 | 23 | 22 |
| 60′ pH5 | 33 | 26 | 26 |
| 75′ pH6.4 | 61 | 36 | 39 |
| 90′ pH6.4 | 61 | 38 | 45 |
| 105′ pH7.4 | 71 | 52 | 67 |
| 120′ pH7.4 | 72 | 54 | 71 |
| 150′ pH8.2 | 90 | 83 | 95 |

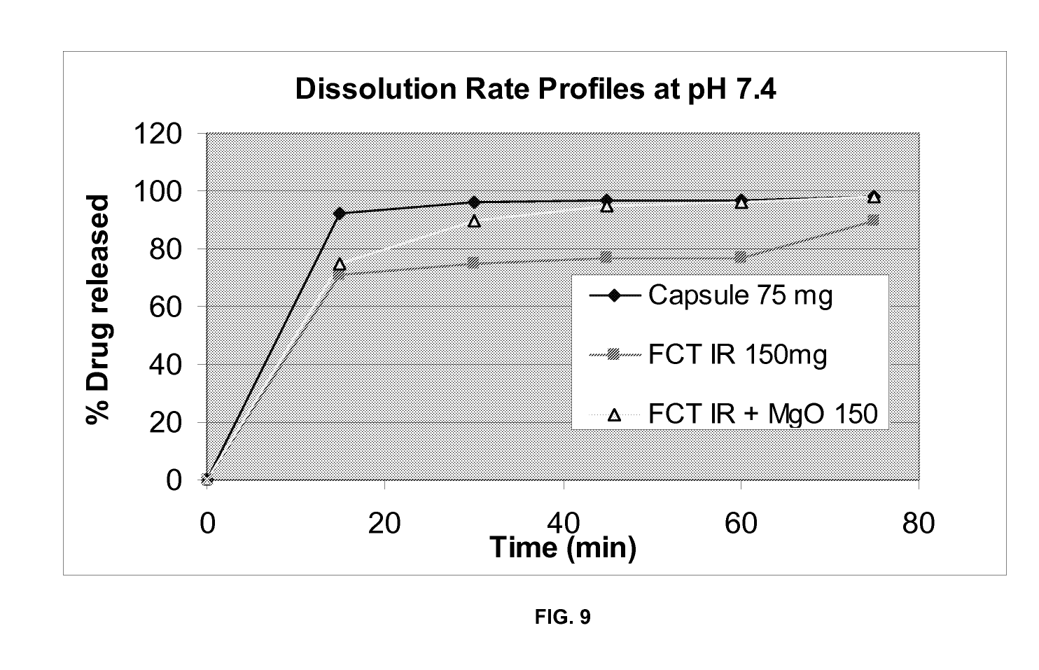

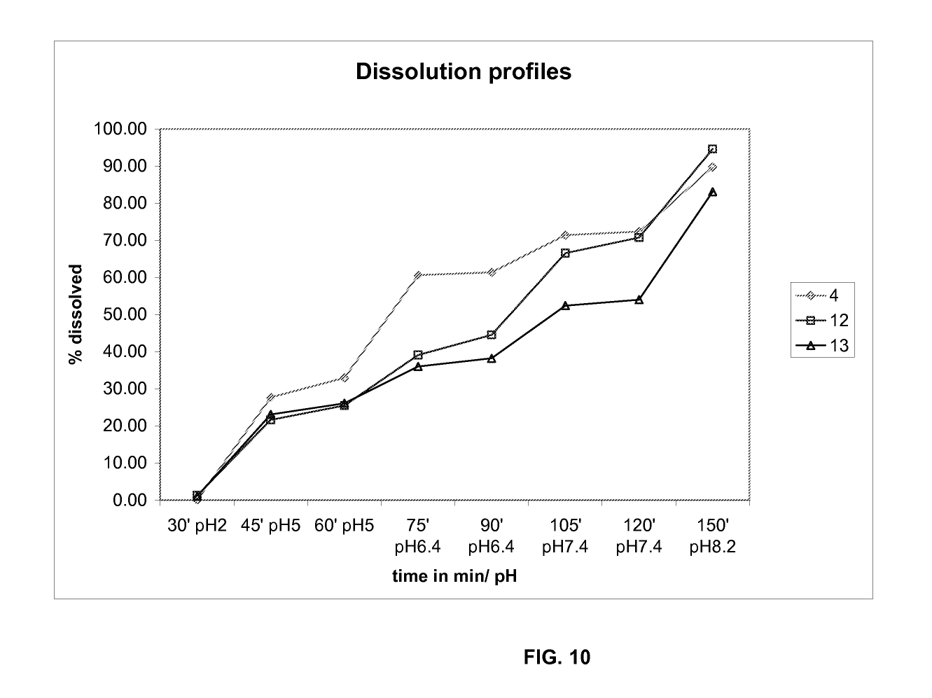

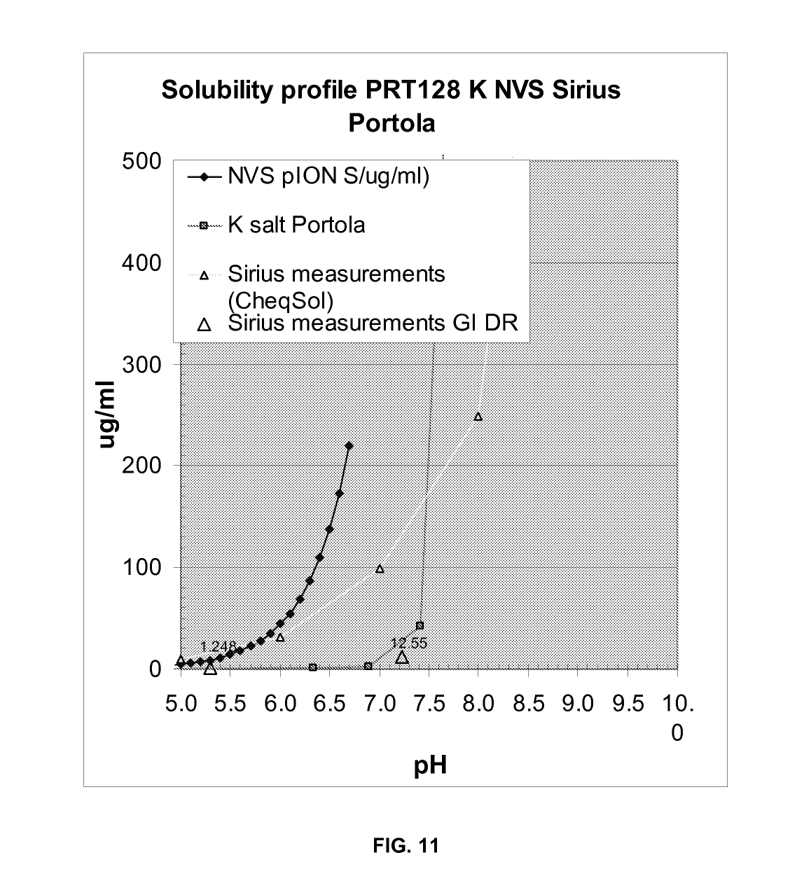

See also FIG. 9 and FIG. 10.

### Example 15

### Testing of Compositions

Compositions were subjected to physical and chemical testing to assess the quality of the granules produced by roller compaction process. The bulk/tapped density was measured according to the USP method. The ATM sonic sifter was used to test the PSD of granules in various stages of processing. The sample size was 5-10 g. The flow properties of the roller compacted lubricated granules were tested using a Sotax flow tester. About 110 g of granules were used for the tests, and standard six pre-vibration/vibration modes were employed. The flow property of the sample was expressed as a flow index, '/ref, and as Carr's Index.

The tablets were tested for dissolution (profile) by conventional dissolution methods (single pH) and multi-stage (multi-pH) dissolution methods. A 5-stage (target pH 2.0, 5.0, 6.4, 7.4, and 8.0, 30 min each stage) method was used for formulation screening. The single stage conventional dissolution method (in pH 7, 4.50 mM phosphate buffer) was used during process and stability evaluation. Physical testing included appearance, weight, weight variation, hardness, and thickness measurements. The following in-process testing equipments were used, as necessary, during the various manufacturing stages: Bulk density/tap density meter, Sonic sifter, Sotax flow tester, Friabilator, Hardness tester, Vernier calipers, Weighing balances and Disintegration Tester USP dissolution apparatus.

The multi-pH method is custom designed using conventional USP Apparatus 2. Starting with pH 2HCl media at 200 ml and paddle 50 rpm condition, the pH are gradually increased by adding higher pH buffers such as acetate buffer, phosphate buffer, and finally sodium hydroxide to achieve target pHs. The volume accordingly increased stepwise to about 900 ml at the last stage.

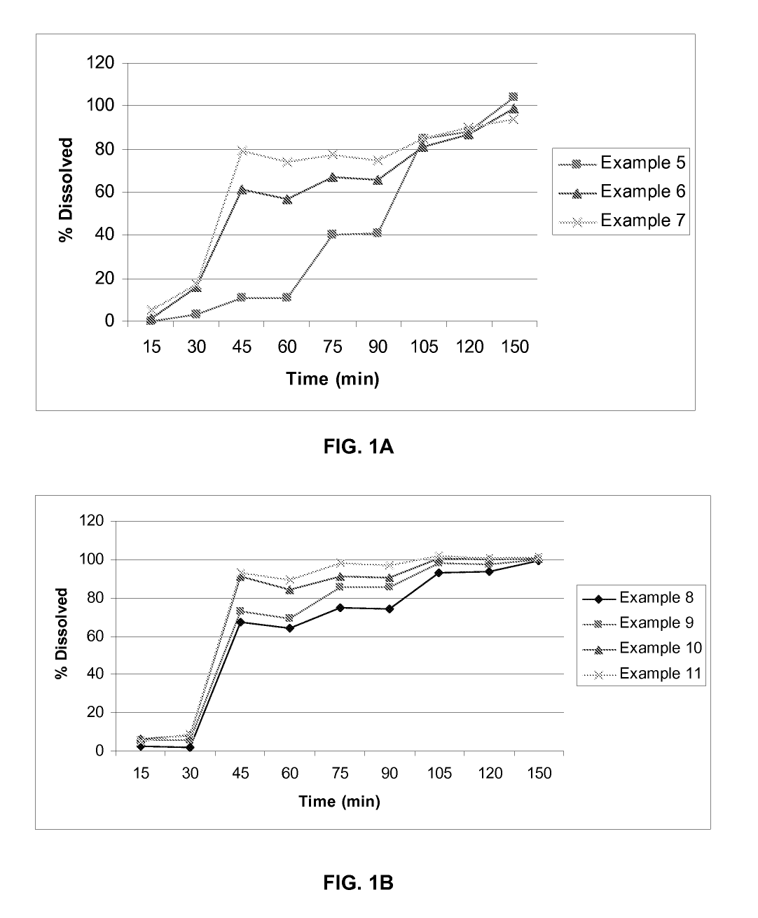

Roller compacted formulations were tested at T=initial, packaged in 75 cc HDPE bottles with 2 desiccant canisters, induction sealed, and placed on stability at 25° C./60% RH, 30° C./65% RH and 40° C./75% RH. The stability testing was conducted according to a pre-established stability protocol. Samples at 40° C./75% RH were pulled at designated time-points and analyzed. Samples were tested for physical appearance (Visual, White to off-white biconvex round tablets), potency, related substances assay, dissolution (Profile) and moisture. This stability study has been completed and up to six months stability data is reported. Multi-pH dissolution profiles for Examples 5-11 are shown in FIGS. 1A and B.

### Example 16

### Effect of Milling Method and Screen Size

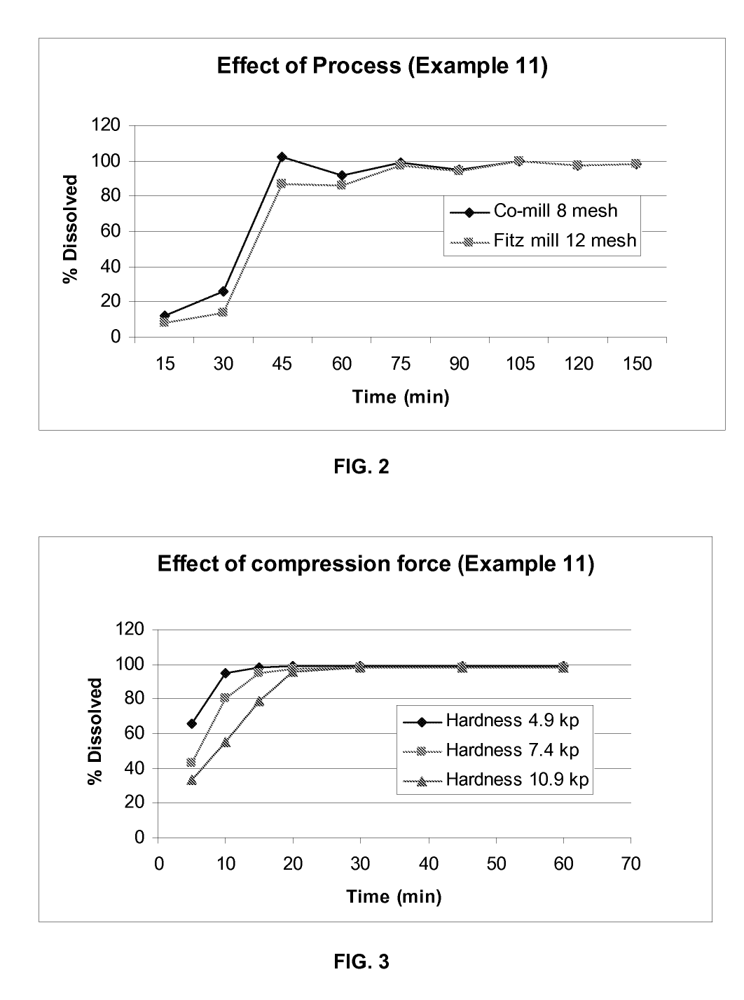

Dissolution results of Example 11 are provided in Table 2 and release profiles are shown in FIG. 2.

TABLE 2
Dissolution Profile (5-stage) for Elinogrel Immediate Release Tablets 100 mg -
Impact of Milling method and screen size post Roller Compaction.

| Test/Methods | | | | | | | | | |
|---|---|---|---|---|---|---|---|---|---|
| Dissolution | | | | | | | | | |
| In multi-pH | | | | | | | | | |
| method | 15 | 30 | 45 | 60 | 75 | 90 | 105 | 120 | 150 |
| (n = 3) | Acid pH 2.0 | | Acid pH 5.0 | | Buffer pH 6.4 | | Buffer pH 7.4 | | Buffer pH 8.2 |
| | 8 Mesh | | | | | | | | |
| Min | 10 | 14 | 90 | 87 | 98 | 94 | 100 | 97 | 97 |
| Max | 16 | 40 | 110 | 96 | 100 | 96 | 101 | 98 | 99 |
| Mean | 12 | 26 | 102 | 92 | 99 | 95 | 100 | 97 | 98 |
| % RSD | 28.7 | 51.2 | 10.7 | 5.0 | 1.0 | 0.8 | 0.7 | 0.8 | 0.9 |
| | 12 Mesh | | | | | | | | |
| Min | 7 | 13 | 85 | 85 | 97 | 94 | 99 | 97 | 97 |
| Max | 9 | 15 | 90 | 87 | 98 | 95 | 100 | 98 | 99 |
| Mean | 8 | 14 | 87 | 86 | 97 | 94 | 100 | 97 | 98 |
| % RSD | 8.8 | 5.3 | 3.7 | 1.3 | 0.5 | 0.6 | 0.5 | 0.7 | 0.7 |

It can be seen that the dissolution profile for Example 5 in multi-pH method was unchanged after the change of process parameters.

### Example 17

### Effect of Tablet Hardness on Dissolution

Most tablets were compressed to a target hardness of 7-9 kP. In Example 11, the tablets were also compressed at 4-6 kP (average 4.9) and 9-12 kP (average 10.9) to assess the impact of hardness on disintegration and dissolution properties using single pH dissolution method. Disintegration results are provided in Table 3 and dissolution results in Table 4. The single-pH dissolution profile was also plotted in FIG. 3.

TABLE 3

| | | |
|---|---|---|
| Test | Example 9 | Example 11 |

| | | | |
|---|---|---|---|
| Individual Weights, | Min | 441.5 | 444.8 |
| mg | Max | 456.8 | 454.9 |
| | Mean | 448.6 | 450.3 |
| Friability, % | | 0.11 | 0.0 |
| Hardness, kP | Min | 5.30 | 6.00 |
| | Max | 7.30 | 7.80 |
| | Mean | 6.48 | 6.85 |
| Thickness, mm | Min | 4.56 | 4.60 |
| | Max | 4.60 | 4.66 |
| | Mean | 4.58 | 4.63 |
| Disintegration, min:sec | | 02:10 | 03:30 |
| Flow angle ratio | | 0.37 | 0.35 |
| (α′/α_{ref}) of Final | | | |
| Blend | | | |

TABLE 4
Dissolution Profile (Single Stage) for Example 11 Immediate Release Tablets 100 mg -
Impact of Hardness

| Test/ | | | | | | | | | | | |
|---|---|---|---|---|---|---|---|---|---|---|---|
| Method | Example 11 | | | | | | | | | | |
| Dissolution | Hardness 4.9 kP | | | | | | | Hardness 7.4 kP | | | |
| Single pH | Time in Minutes | | | | | | | Time in Minutes | | | |
| method | Buffer pH 7.4 | | | | | | | Buffer pH 7.4 | | | |
| (n = 6) | 5 | 10 | 15 | 20 | 30 | 45 | 60 | 5 | 10 | 15 | 20 |
| Min | 62 | 94 | 97 | 97 | 97 | 97 | 97 | 40 | 69 | 91 | 94 |
| Max | 68 | 96 | 99 | 100 | 100 | 100 | 100 | 50 | 89 | 99 | 99 |
| Mean | 66 | 95 | 98 | 99 | 99 | 99 | 99 | 43 | 80 | 95 | 97 |
| SD | 2.8 | 0.6 | 1.2 | 1.4 | 1.3 | 1.3 | 1.4 | 4.3 | 10.4 | 3.5 | 2.1 |

| Test/ | | | | | | | | | | |
|---|---|---|---|---|---|---|---|---|---|---|
| Method | Example 11 | | | | | | | | | |
| Dissolution | Hardness 7.4 kP | | | Hardness 10.9 kP | | | | | | |
| Single pH | Time in Minutes | | | Time in Minutes | | | | | | |
| method | Buffer pH 7.4 | | | Buffer pH 7.4 | | | | | | |
| (n = 6) | 30 | 45 | 60 | 5 | 10 | 15 | 20 | 30 | 45 | 60 |
| Min | 96 | 96 | 96 | 29 | 50 | 73 | 94 | 96 | 97 | 97 |
| Max | 99 | 100 | 100 | 38 | 66 | 91 | 98 | 99 | 99 | 99 |
| Mean | 98 | 98 | 98 | 33 | 55 | 79 | 96 | 98 | 98 | 98 |
| SD | 1.5 | 1.5 | 1.4 | 3.8 | 7.1 | 8.2 | 1.5 | 1.3 | 1.2 | 1.2 |

The compression force has an impact on tablet disintegration and drug dissolution, i.e., a slightly faster disintegration and release was obtained with tablets manufactured with lower hardness. However, complete dissolution in a single pH method was achieved within 20 min for the entire hardness range (4-12 kP). Based on the minimal effect of milling method and screen size for Example 5, as shown above, it is anticipated that the dissolution profile for low and high hardness range using multi-pH method is not affected.

### Example 18

### Stability

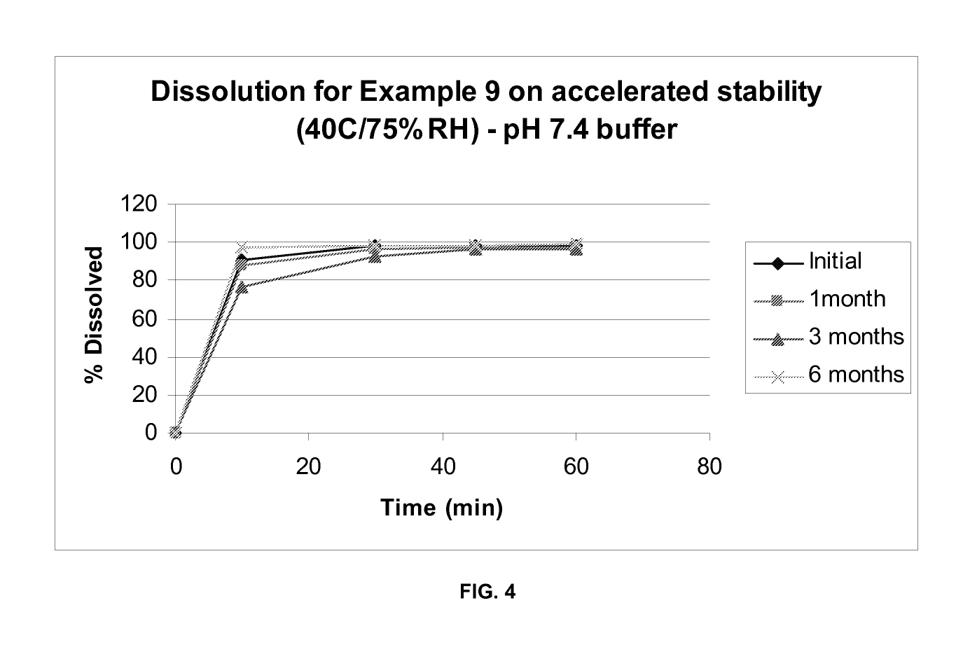

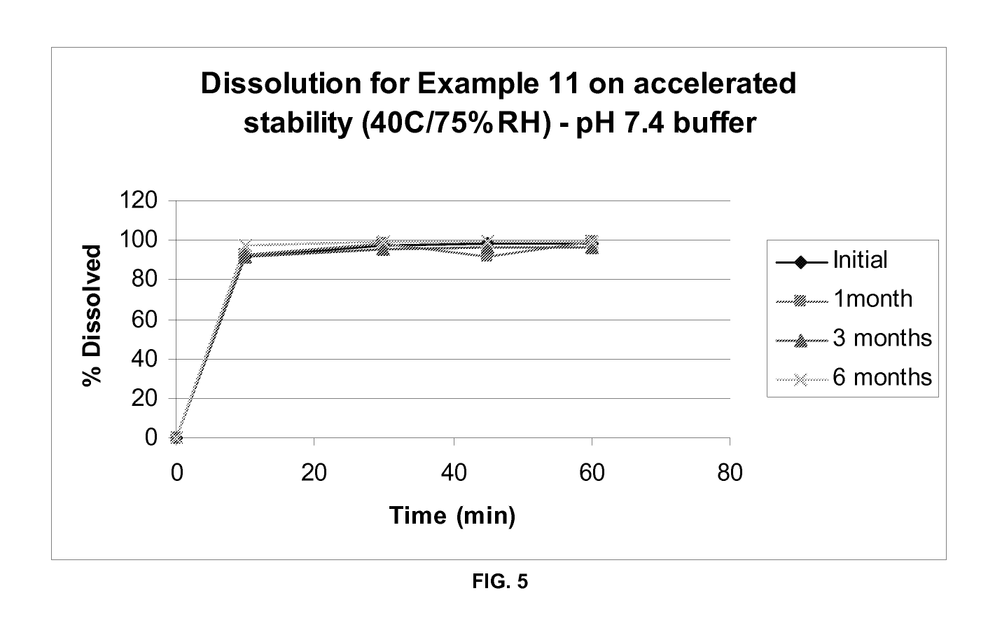

Examples 9 and 11 were selected for accelerated stability evaluation based on their superior dissolution property. The stability results are provided in Tables 5 and 6 and dissolution profiles are provided in FIGS. 4 and 5 for stability prototype batches Examples 9 and 11, respectively.

TABLE 5
Stability Results for Elinogrel Immediate Release Tablets 100 mg - Example 9

| | Example 9 | | | | | | | | | | | | | | | |
|---|---|---|---|---|---|---|---|---|---|---|---|---|---|---|---|---|
| | | | | | T = 1 month | | | | T = 3 months | | | | T = 6 Months | | | |
| Test/Method | T = initial | | | | 40° C./75% RH | | | | 40° C./75% RH | | | | 40° C./75% RH | | | |
| Physical | Off-white biconvex | | | | Off-white biconvex | | | | Off-white biconvex | | | | Off-white biconvex | | | |
| Appearance/ | round tablets | | | | round tablets | | | | round tablets | | | | round tablets | | | |
| Visual | | | | | | | | | | | | | | | | |
| Moisture, % w/w/ | 3.4 | | | | 3.0 | | | | 3.7 | | | | 3.6 | | | |
| USP<921> | | | | | | | | | | | | | | | | |
| method a | | | | | | | | | | | | | | | | |
| Potency, % | 1: 97.7 | | | | 1: 98.0 | | | | 1: 94.6 | | | | 1: 96.6 | | | |
| | 2: 98.1 | | | | 2: 97.8 | | | | 2: 95.1 | | | | 2: 96.4 | | | |
| | Mean: 97.9 | | | | Mean: 97.9 | | | | Mean: 94.9 | | | | Mean: 96.5 | | | |
| Total related | 0.40 | | | | 0.41 | | | | 0.42 | | | | 0.49 | | | |
| substances | | | | | | | | | | | | | | | | |
| Dissolution, % in | Minutes | | | | Minutes | | | | Minutes | | | | Minutes | | | |
| single pH | | | | | | | | | | | | | | | | |
| dissolution | | | | | | | | | | | | | | | | |
| method | | | | | | | | | | | | | | | | |
| | Buffer pH 7.4 | | | | Buffer pH 7.4 | | | | Buffer pH 7.4 | | | | Buffer pH 7.4 | | | |
| (n = 6) | 10 | 30 | 45 | 60 | 10 | 30 | 45 | 60 | 10 | 30 | 45 | 60 | 10 | 30 | 45 | 60 |
| Min | 87 | 97 | 97 | 97 | 85 | 94 | 95 | 95 | 64 | 87 | 94 | 94 | 95 | 96 | 96 | 96 |
| Max | 94 | 99 | 99 | 99 | 90 | 99 | 99 | 100 | 87 | 98 | 99 | 99 | 99 | 100 | 100 | 100 |
| Mean | 91 | 98 | 98 | 98 | 88 | 96 | 97 | 97 | 77 | 93 | 96 | 96 | 97 | 98 | 98 | 99 |
| SD | 2.9 | 0.7 | 0.6 | 0.7 | 2.4 | 1.8 | 1.9 | 1.9 | 9.9 | 3.9 | 2.0 | 1.7 | 1.3 | 1.4 | 1.5 | 1.5 |

TABLE 6
Stability Results for Elinogrel Immediate Release Tablets 100 mg - Example 11

| | Example 11 | | | | | | | | | | | | | | | |
|---|---|---|---|---|---|---|---|---|---|---|---|---|---|---|---|---|
| | | | | | T = 1 month | | | | T = 3 months | | | | T = 6 Months | | | |
| Test/Method | T = initial | | | | 40° C./75% RH | | | | 40° C./75% RH | | | | 40° C./75% RH | | | |
| Physical | Off-white biconvex | | | | Off-white biconvex | | | | Off-white biconvex | | | | Off-white biconvex | | | |
| Appearance/ | round tablets | | | | round tablets | | | | round tablets | | | | round tablets | | | |
| Visual | | | | | | | | | | | | | | | | |
| Moisture, % w/w/ | 3.4 | | | | 3.3 | | | | 3.8 | | | | 3.9 | | | |
| USP<921> method a | | | | | | | | | | | | | | | | |
| Potency, %/ | 1: 97.2 | | | | 1: 98.0 | | | | 1: 95.6 | | | | 1: 97.3 | | | |
| | 2: 97.4 | | | | 2: 98.2 | | | | 2: 95.4 | | | | 2: 97.2 | | | |
| | Mean: 97.3 | | | | Mean: 98.1 | | | | Mean: 95.5 | | | | Mean: 97.3 | | | |
| Total related | 0.38 | | | | 0.40 | | | | 0.39 | | | | 0.46 | | | |
| substances | | | | | | | | | | | | | | | | |
| Dissolution, % in | Minutes | | | | Minutes | | | | Minutes | | | | Minutes | | | |
| single pH | | | | | | | | | | | | | | | | |
| dissolution method | | | | | | | | | | | | | | | | |
| | Buffer pH 7.4 | | | | Buffer pH 7.4 | | | | Buffer pH 7.4 | | | | Buffer pH 7.4 | | | |
| (n = 6) | 10 | 30 | 45 | 60 | 10 | 30 | 45 | 60 | 10 | 30 | 45 | 60 | 10 | 30 | 45 | 60 |
| Min | 86 | 90 | 91 | 91 | 89 | 95 | 96 | 96 | 89 | 95 | 95 | 95 | 95 | 97 | 97 | 97 |
| Max | 98 | 100 | 102 | 102 | 99 | 100 | 101 | 101 | 95 | 96 | 98 | 98 | 99 | 101 | 101 | 102 |
| Mean | 92 | 97 | 98 | 98 | 93 | 98 | 99 | 99 | 92 | 95 | 96 | 96 | 97 | 99 | 99 | 99 |
| SD | 4.4 | 4.0 | 4.1 | 4.1 | 3.7 | 2.0 | 1.7 | 1.7 | 2.2 | 0.8 | 1.4 | 1.4 | 1.7 | 1.6 | 1.8 | 1.8 |

The physical appearance, potency, related substance, moisture, and potency were satisfactory up to the 6 months test-point. The dissolution release profiles for Examples 9 and 11 were also satisfactory. Stability data indicates that examples are stable when stored at 40° C./75% RH for up to 6 months.

### Example 19

### Pharmacokinetic Studies 1

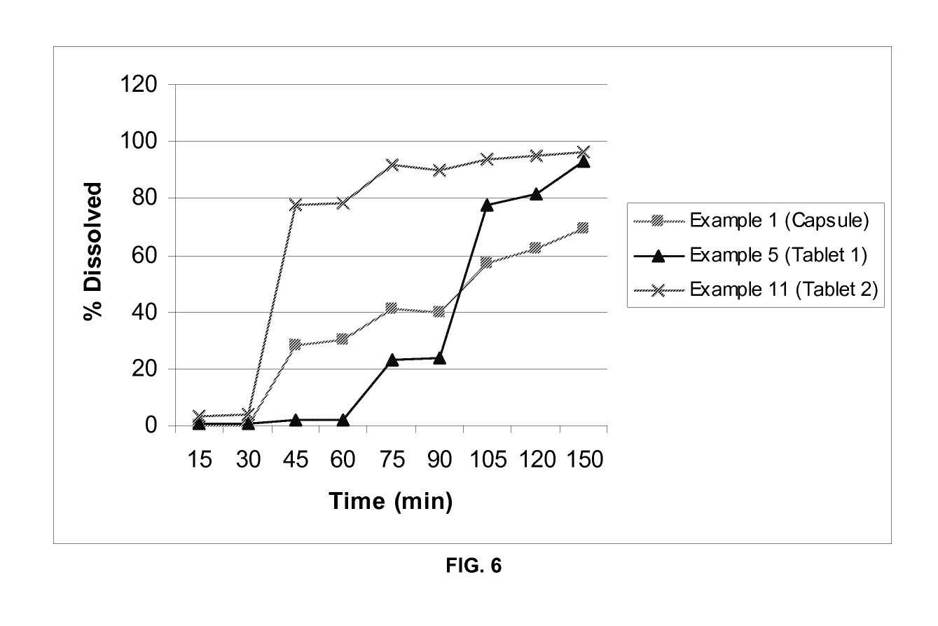

Three immediate release formulations were selected for relative pharmacokinetic comparison study: i.e., examples 1, 5, and 11. The multi-stage dissolution profiles of the three formulations are provided in FIG. 6. A single dose of capsules (2 units of 50 mg each) and tablets each containing 100 mg elinogrel was administered on 3 separate occasions to 16 healthy males and/or healthy, non-pregnant, non-lactating, females aged 18 to 65 years with a Body Mass Index (BMI) of 19-35 kg/m2 (inclusive) and estimated or measured weight ≧55 kg in a randomized order in the fasted state.

### Food and Fluid Intake

Subjects were provided with meals at normal times on Day −1 of each study period. Subjects were provided with a light snack at approximately 23:30 and fasted from all food and drink (except water) from midnight on the day prior to dosing until approximately 4 hours post-dose at which time lunch was provided. An evening meal was provided at approximately 9 hours post dose and an evening snack at 14 hours post dose. On subsequent days, meals were provided at normal times. All meals were at the same for all subjects during all three dosing periods to eliminate any possible food effect on the evaluation of pharmacokinetics. Subjects were allowed water up to 2 hours before the scheduled dosing time and were provided with 240 mL of water at 2 hours post dose. Decaffeinated fluids were allowed ad libitum after lunch on the day of dosing. If, for technical reasons, dosing was delayed for more than two hours beyond the expected dosing time, subjects received 200 mL Lucozade Sport at the originally scheduled dosing time, or earlier if possible.

### Administration of Test Preparations

Each subject will receive a single dose of one of the formulations on 3 separate occasions. Dosing will commence at approximately 08:00. Each preparation will be administered orally with 240 mL water.

### Pharmacokinetic Blood Sampling

Venous blood samples (2 mL) were withdrawn via an indwelling canella or by venepuncture according to the following time schedule: 0 (pre dose) and at 0.5, 1, 1.5, 2, 3, 4, 5, 6, 8, 10, 12, 16, 24, 48 and 72 hours post-dose (n=16). Samples collected at 48 and 72 hours were as outpatient visits. The samples were centrifuged at approximately 1600 g for 20 minutes at 4° C. within 20 minutes of the time of blood collection. The resulting plasma fraction were split into two equal aliquots of approximately 0.5 mL each and frozen in labeled polypropylene Microfuge tubes at approximately minus 20° C. until the end of each study period and then at minus 70° C. or below until required for assay.

Discharge from the Clinical Unit

A subject was allowed to leave the premises at: 24 hours post dose following completion of study specific procedures, providing that:

1) no AEs had been reported during the study visit; and
2) the subject responded in the affirmative when asked if he or she is feeling well.
If any of these conditions is not met, then the subject will only be allowed to leave the premises once declared fit to do so by a fully registered physician. Volunteers returned to the unit at 48 and 72 hours post dose to provide further plasma samples.

### Post-Study Measurements

A follow-up assessment of all subjects was made within the 5-10 day period following the final dose. This included the following:

1) physical examination;
2) vital signs measurements (blood pressure and pulse rate after 5 min supine);
3) ECG (using MAC 5500) after 5 minutes supine;
4) clinical laboratory tests;
5) female subjects had a urine pregnancy test.

Subjects were discharged from the study when they have attended their post study medical examination.

### Pharmacokinetic Data Analysis

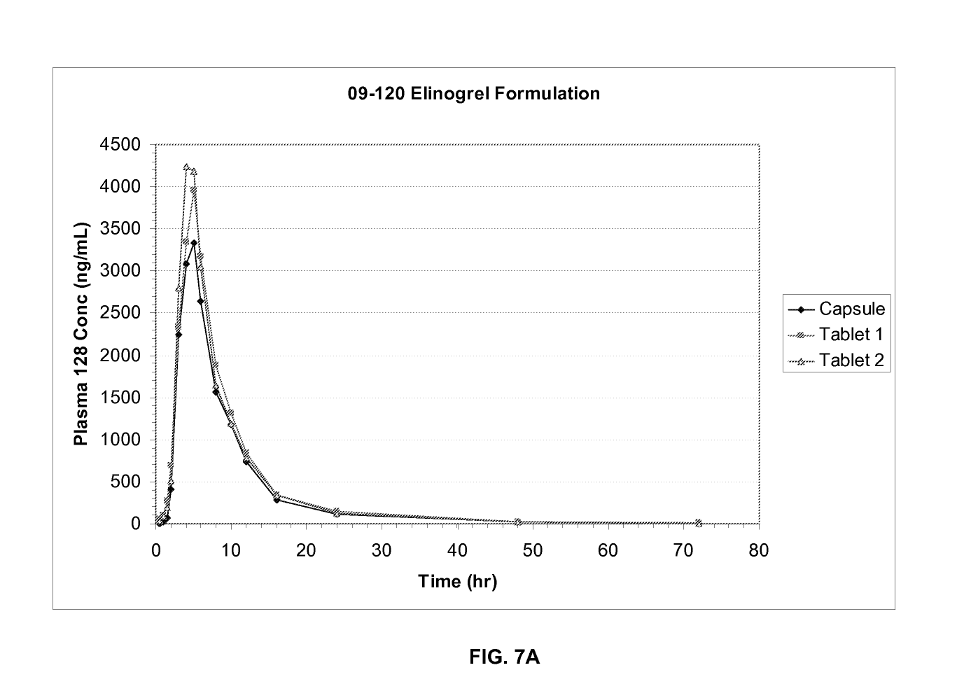

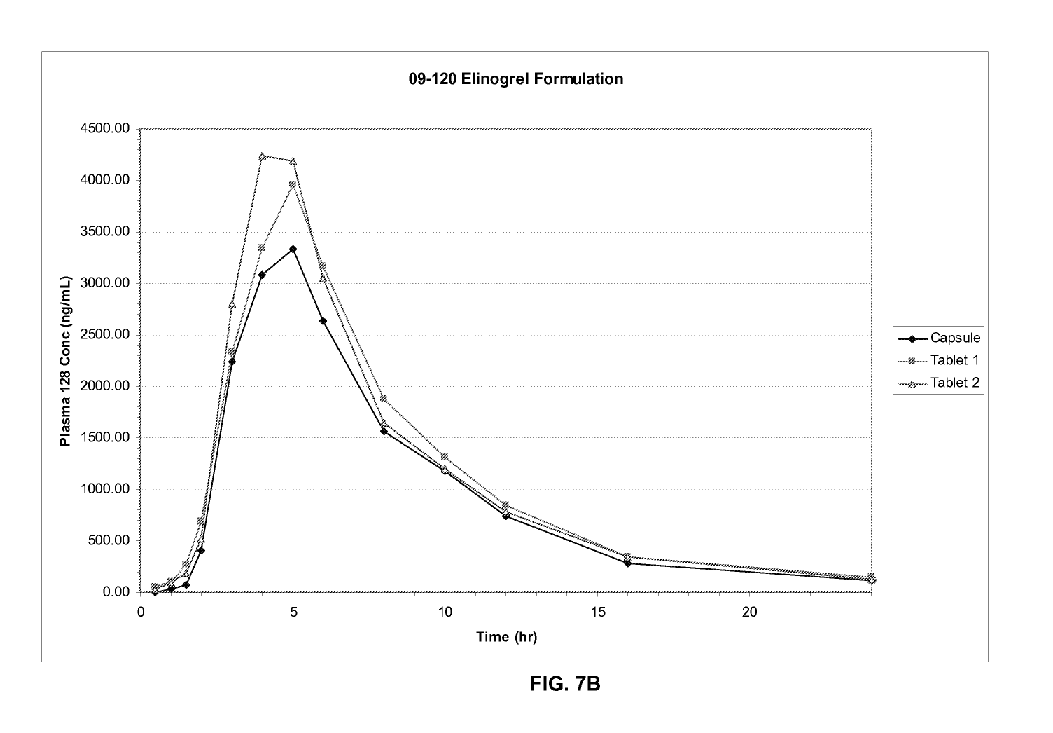

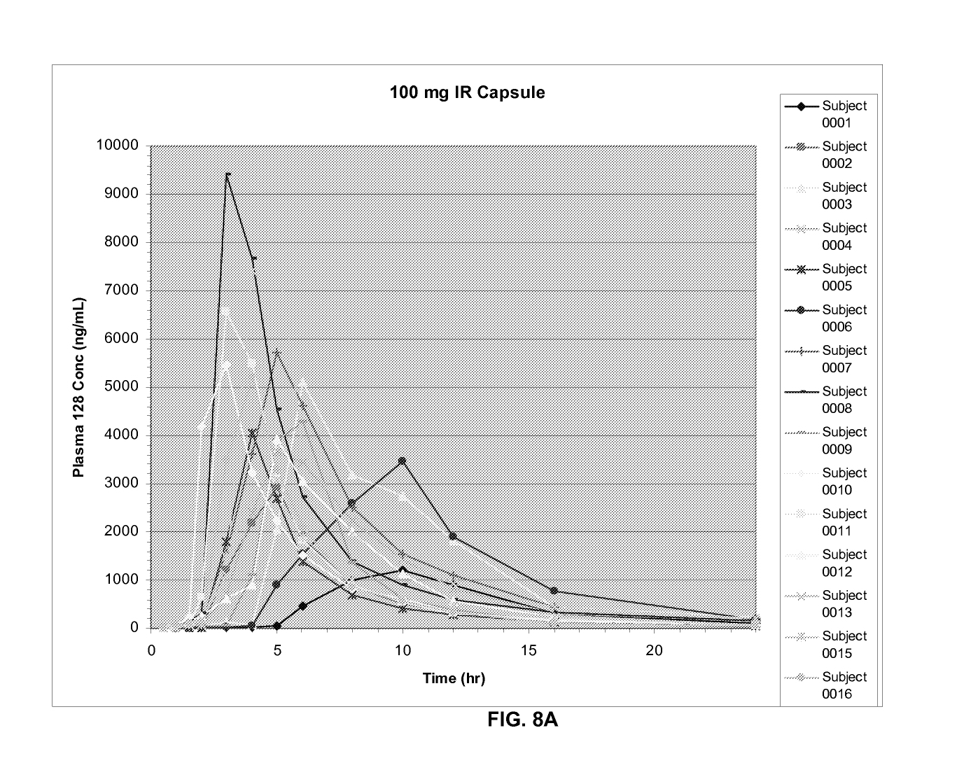

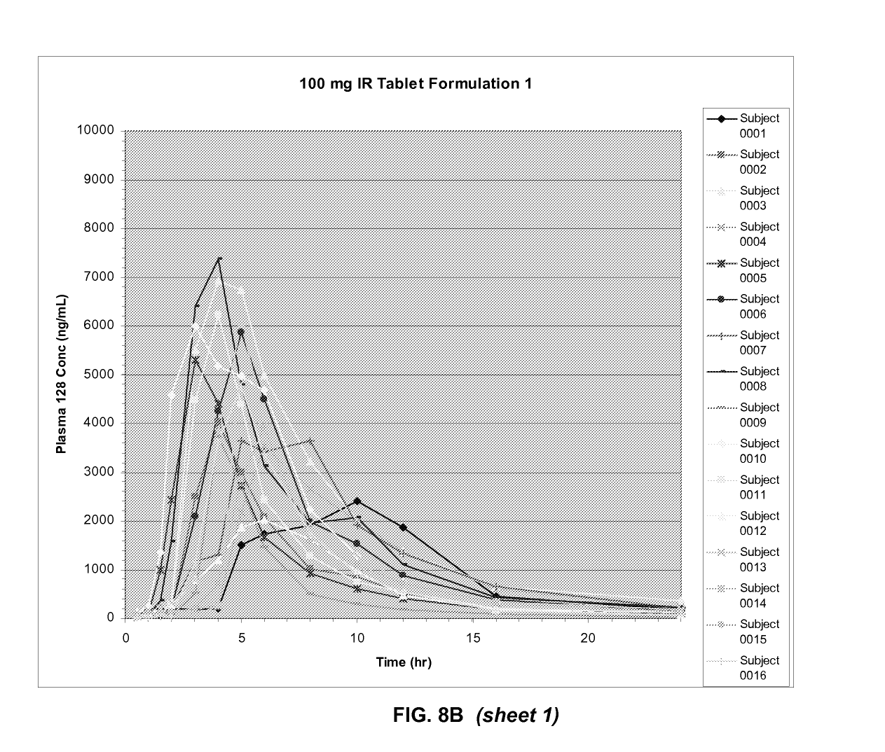

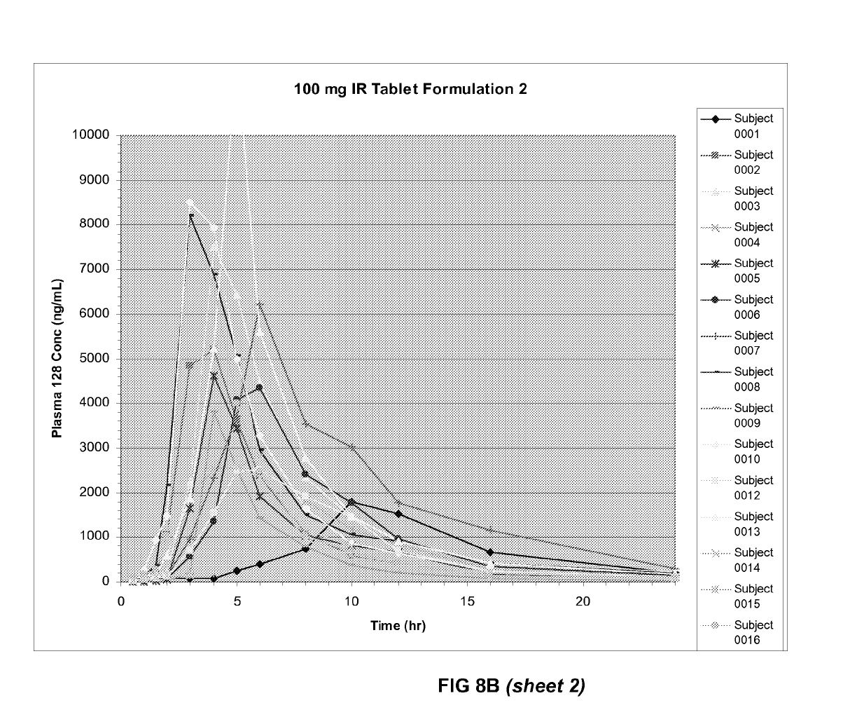

Plasma samples were analysed for elinogrel by the following procedures. This quantitative study describes the pharmacokinetic profile of elinogrel. For a study of this type, data sets of 12 were considered appropriate to describe the pharmacokinetic parameters. The plasma concentration data for elinogrel was provided by CEDRA Corporation analysed by Quotient Clinical, using WinNonlin ver 5.0.1, Pharsight, Inc. Actual rather than nominal times were used for all PK analyses. Plasma concentration data was tabulated and plotted for each subject for whom concentrations are quantifiable. Pharmacokinetic analysis of the concentration time data obtained was performed using appropriate non-compartmental techniques to obtain estimates of the following pharmacokinetic parameters;
1) Maximal plasma concentration (Cmax);
2) Time to reach Cmax(tmax);
3) Time to first quantifiable plasma concentration (tlag);
4) Area under the concentration-time curve (AUC) from dosing to infinity (AUC 0∞);
5) AUC from dosing to last measured time point (AUC 0-t);
6) Mean residence time (MRT)
7) Percentage AUC extrapolated beyond last measured time point;
8) Relative bioavailability of test formulation to reference (Frel);
9) Terminal elimination rate constant (λa);
10) Terminal half-life (t1/2).
   Descriptive summary statistics (N, mean, standard deviation, median, range) were presented for baseline demographics and safety fields, including changes from baseline as appropriate. The mean plasma concentration-time profiles of three formulations are provided in FIG. 7. The individual plasma concentration-time profiles for each formulation are provided in FIG. 8a-c, respectively. Pharmacokinetic results are provided in Table 7.

TABLE 7
Pharmacokinetic results (GeoMean + CV %)

| | Reference IR | IR Tablet 1 | IR Tablet 2 |
|---|---|---|---|
| PK parameter | (Example 1) | (Example 5) | (Example 11) |
| Cmax (ng/mL) | 4353 (52%) | 4555 (38%) | 4994 (60%) |
| tmax (h) | 4.92 (38%) | 4.73 (30%) | 4.61 (34%) |
| AUC∞ | 23740 (39%) | 27400 (38%) | 28560 (38%) |
| (ng · h/mL) | | | |
| C24 (ng/mL) | 97.25 (70%) | 120.6 (76%) | 107.0 (80%) |
| Cmax/C24 ratio | 44.8 | 37.8 | 46.7 |
| MRT (h) | 9.48 (23%) | 9.55 (20%) | 9.30 (25%) |
| t½ (h) | 9.95 (16%) | 10.15 (15%) | 9.61 (20%) |
| Frel (%) | — | 123.6 (30%) | 121.0 (25%) |

### Example 19

### Pharmacokinetic Studies 2

This study employed an open-label, randomized, four-treatment, single-dose, four-period, crossover design in healthy male and female subjects. A total of 50 subjects were enrolled and randomized to receive one of four sequences of treatments, approximately 40 subjects were required to complete all treatment periods. Each subject participated in a screening period (from 21 days prior to dosing up to 2 days prior to dosing), a baseline visit for each period (at least 12 hours preceding the dose administration), and single-dose treatment periods. Each treatment period comprised of a dosing day and a PK assessment period up to 72 hours post-dose. An end-of-study evaluation was completed after Period 5, Day 8, or at early termination. Between each dose administration, there was a washout period of at least 5 days. This washout period was based on the half-life of elinogrel (range of mean values: 9 to 16 hours) and the known pharmacokinetic inter- and intra-subject variability associated with elinogrel.

Subjects received the following treatments in a randomized order:

Treatment with IR tablet of Example 11 containing pH-modifying alkalizing agent, 150 mg.

Treatment with IR tablet of Example 12 tablet, 150 mg.

Treatment with IR capsule of Example 4, 2×75 mg.

Treatment with IR capsule of Example 1 or 2 (Reference): 2×75 mg.

At each baseline (Day −1), subjects fasted overnight (for at least 10 hours prior to dosing) and continued to fast until 4 hours post-dose. Pharmacokinetic assessments were performed starting at pre-dose (Oh, Day 1) and continuing up until 72 hours post-dose. Subjects were domiciled at least 12 hours prior to dosing through to at least 24 hours post-dose for PK sample collection, at which time they were discharged from the study center. Safety assessments were performed up to 48 hours post-dose. Subjects then returned to the study center on the next 2 mornings to complete the remaining PK sample collection period (i.e., 48 and 72 hours post-dose samples). Study completion evaluations were performed following the 72-hour pharmacokinetic blood draw in the last treatment period, or in case of early termination. Safety assessments included physical examinations, ECGs, vital signs, standard clinical laboratory evaluations hematology, blood chemistry, urinalysis, adverse event and serious adverse event monitoring.

After oral administration of a single dose of elinogrel formulations the median Tₘₐₓ was around 5 hours post-dose in all treatment groups as shown in Table 35. Mean exposure (Cmax and AUC) of elinogrel was highest when elinogrel was administered as MF capsule formulation (R). Exposure was similar for IR tablet containing pH modifying agent (T1), IR tablet (T3) and IR capsule (T4). The mean half-life ranged from 7-8 hours for all formulations and CL/F and Vz/F was highly variable as shown in Table 8.

TABLE 8
Plasma PK parameters of elinogrel following oral administration of a single 150 mg
dose of elinogrel IR tablet with pH-modifier, standard IR tablet, IR capsule matching Portola
MF capsule and Portola MF capsule formulation

| Treatment | Tmax | Cmax | AUClast | AUCinf | CL/F | Vz/F | T½ |
|---|---|---|---|---|---|---|---|
| Group | (hr) | (ng/mL) | (hr * ng/mL) | (hr * ng/mL) | (L/hr) | (L) | (hr) |
| T1 | 5.00 | 2304 | 16730 | 17220 | 11.2 | 107 | 7.33 |
| (N = 37-38) | (3.0; 10) | (767) | (7842) | (7861) | (6.44) | (46.4) | (2.25) |
| T3 | 5.00 | 2195 | 17000 | 17430 | 14.6 | 118 | 7.03 |
| (n = 39-40) | (3.0; 10.0) | (1448) | (10792) | (10980) | (17.2) | (74.8) | (2.43) |
| T4 | 5.00 | 2303 | 16590 | 16900 | 16.6 | 137 | 7.18 |
| (N = 40) | (2.0; 8.0) | (1135) | (10173) | (10198) | (26.1) | (124) | (2.17) |
| R | 5.00 | 4201 | 27590 | 27950 | 8.30 | 83.5 | 8.02 |
| (n = 39) | (2.0; 8.0) | (2323) | (15399) | (15402) | (7.08) | (58.1) | (2.88) |

Mean and SD are presented for all parameters except for Tmax in which median values are shown
T1 = IR tablet with pH-modifier (150 mg),
T3 = IR tablet (150 mg),
T4 = IR capsule (2 × 75 mg),
R = MF capsule (2 × 75 mg)
AUCinf, CL/F, Vz/F, T½ not calculated as r² adjusted <0.75 for 1 subject in treatment group T1 and T3.

### Example 20

### Wettability

TABLE 9
Contact Angle Measurement Results

| Polymorph | n = | 0 sec | 10 sec | 20 sec | 30 sec | 60 sec |
|---|---|---|---|---|---|---|
| Free acid | 2 | 65.9 | | | 60.8 | 60.1 |
| B | 2 | 72.4 | | | 54.6 | 53.5 |
| A | 2 | 78.8 | | | 53.3 | 51.0 |
| B | 2 | 79.7 | | | 55.2 | 52.8 |

The contact angle measurements show the significant difference in the surface hydrophobicity between polymorph B and A, which are are hydrophobic, and polymorph poly-D, which is hydrophilic.

The critical angle is estimated around 50° (>50° hydrophobic; <50° hydrophilic) according to BC Lippold, A. Ohm, Correlation between wettablity and dissolution rate of pharmaceutical powders, Internatioal Journal of Pharmaceutics, 28 (1986) 67-74.

### Method

The contact angle instrument was EasyDrop DSA15E (Krüss, Hamburg, Germany). The syringe was a NE44, with a needle diameter of 0.5 mm (Krüss, Hamburg, Germany). The standards (Krüss, Hamburg, Germany) were CP24 20007107, contact angle Gauge#4=30.00°±0.3, Gauge#5=60.00°±0.3, Gauge#6=120.00°±0.3.

The powder was pressed (approx 50 mg powder, 3 tons, 2 min) in tablets and analyzed by contact angle measuremnt. The angle between the solid and the liquid is measured after 0 and up to 60 seconds using water.

All determinations used the above method, and the limit for the standards measurements are well within the given limits.

### Example 21

### Surface Tension

### Method: Microtensiometry

Surface tension is measured with the Kibron Delta-8 tensiometer which is an 8-channel microbalance, meaning that 8 samples can be measured simultaneously. Samples are prepared in standard footprint 96-well plates using 50 μL sample volumes. The technique measures the weight of the meniscus using a high performance micro balance. A thin rod is immersed into the sample and then pulled out, and the maximum force is measured (also known as the Du-Nouy-Padday method, J. Chem. Soc., Faraday Trans. 1, 1975, 71, 1919-1931, DOI:10.1039/F19757101919). The weight of the meniscus depends only on surface tension, rod diameter, and the density of the liquid. Calibration is performed using a liquid with a known surface tension, e.g., deionised water.

Surfactants/amphiphilic molecules contain both hydrophilic and hydrophobic parts. The hydrophilic part of the molecule prefers to interact with water while the hydrophobic part is repelled from water. These surface-active molecules absorb at the air/water interface, decreasing surface tension. As the interface becomes saturated, the molecules start to form aggregates or micelles in the bulk of the liquid with the surface tension remaining constant. The Kibron Delta-8 records the surface tension for a serial dilution of a sample from the lowest to highest concentration, giving the concentration of the surfactant required for CMC, critical micelle concentration. The critical micelle concentration (CMC) results in a sharp transition above which the concentration of the free surfactant/amphiphile molecules remains constant. No further reduction in surface tension with increasing concentration occurs resulting in a plateau in the surface tension vs. concentration curve.

Stock solutions of sample of the potassium salt of formula (I) were prepared in de-ionised water at concentrations of 1.62 mg/mL and 1.81 mg/mL. These stock solutions were used to prepare dilution series for the measurement plate. The study plate has 8 rows (labeled A through G) and 12 columns (labeled 1 through 12), having 96 wells in total. By convention, a cell's position in the plate is designated by its row and column (e.g., position A3, or cell A3, denotes the cell at row A, column 3).

Row A contained a known surfactant prepared in deionized water and diluted twofold in each subsequent column. The concentration of cell A1 was 25 mM; A2, 12.5 mM; A3, 6.25 mM with another twofold dilution for each column up to well A11 at 0.024 mM. Position A12 contained de-ionized water.

Row B contained an in-house QC sample prepared in pH 2 phosphate buffer and diluted twofold in each subsequent column. The concentration of cell B1 was 100 mM; B2, 50 mM; B3, 25 mM with another twofold dilution for each column up to cell B11 at 0.098 mM. Position B12 contained pH 2 phosphate buffer.

Row C contained solutions of the elinogrel potassium salt prepared from the 1.62 mg/mL stock. Position C1 contained 1.62 mg/mL of elinogrel potassium salt in deionized water. Position C2 was a twofold dilution into pH 6 phosphate buffer, i.e., 0.81 mg/mL. The remaining wells were further twofold dilutions into pH 6 phosphate buffer: C3 was 0.40 mg/mL; C4, 0.20 mg/mL with another twofold dilution for each column up to cell C11 at 0.0016 mg/mL. Position C12 contained pH 6 phosphate buffer.

Row D was a repeat of Row C and contained solutions of the potassium salt of formula (I) prepared from the 1.62 mg/mL stock. Position D1 contained 1.62 mg/mL the potassium salt of formula (I) in deionized water. Position D2 was a twofold dilution into pH 6 phosphate buffer, i.e., 0.81 mg/mL. Like row C, the remaining wells were further twofold dilutions into pH6 phosphate buffer from D3 at 0.40 mg/mL up to cell D11 at 0.0016 mg/mL. Position D12 contained pH6 phosphate buffer.

Row E contained solutions of the potassium salt of formula (I) prepared from the 1.81 mg/mL stock. Position E1 contained 1.81 mg/mL of the potassium salt of formula (I) in deionized water. Position E2 was a twofold dilution into pH 9.5 borate buffer, i.e., 0.91 mg/mL. The remaining wells were further twofold dilutions into ph 9.5 borate buffer: Cell E3 was 10 0.45 mg/mL; E4, 0.23 mg/mL; with another twofold dilution for each column up to cell E11 at 0.0018 mg/mL. Position E12 contained pH9.5 borate buffer.

Row F was a repeat of Row E and contained solutions of the potassium salt of formula (I) prepared from the 1.81 mg/mL stock. Position F1 contained 1.81 mg/mL of the potassium salt of formula (I) in deionized water. Position F2 was a twofold dilution into pH 9.5 borate buffer, 15 i.e., 0.91 mg/mL. The remaining wells were further twofold dilutions into pH 9 borate buffer from F3 at 0.45 mg/mL and F4 at 0.23 mg/mL up to cell F11 at 0.0018 mg/mL. Position F12 contained pH9.5 borate buffer.

Row G was a repeat of Row A and contained a known surfactant prepared in deionized water and diluted twofold in the following concentrations: Cell G1 was 25 mM; G2, 12.5 mM; G3, 6.25 mM; with another twofold dilution for each column up to cell G11 at 0.024 mM. Position G12 contained de-ionised water.

Row H contained deionized water as a control.

### Results:

TABLE 10
Results of pH-dependent surface tension measurements

| | | | | | CMC | Surface |
|---|---|---|---|---|---|---|
| | | | | | (mg/ml) | tension |
| Surface | | | | | Limit | at CMC |
| Active | T/° C. | Medium | Method | pH | <1 mg/ml | mN/m |
| Yes | 24.4-24.6 | pH 6.0 | Micro- | 6 | 0.1 | 62 |
| | | phosphate | tensiometer | | | |
| | | buffer | | | | |
| Yes | 24.1 | pH 6.5 | Micro- | 6.5 | >1.7 | 68 |
| | | phosphate | tensiometer | | | |
| | | buffer | | | | |
| Yes | 24.1 | pH 7.0 | Micro- | 7 | >2.7 | 65 |
| | | phosphate | tensiometer | | | |
| | | buffer | | | | |
| Yes | 24.1 | pH 7.5 | Micro- | 7.5 | >4.0 | 63 |
| | | phosphate | tensiometer | | | |
| | | buffer | | | | |
| Yes | 24.1 | pH 8.0 | Micro- | 8 | >3.5 | 63 |
| | | phosphate | tensiometer | | | |
| | | buffer | | | | |
| No | 24.4-24.6 | pH 9.5 | Micro- | 9.5 | n/a | |
| | | borate | tensiometer | | | |
| | | buffer | | | | |
| No | 24.4-24.6 | De- | Micro- | | n/a | |
| | | ionised | tensiometer | | | |
| | | water | | | | |

The potassium salt of formula (I) has no surface activity at pH 9.5 in borate buffer. The potassium salt of formula (I) has no surface activity when prepared in deionized water at concentrations of 1.6-1.8 mg/mL. The natural pH of these solutions is estimated as approximately pH 8. The potassium salt of formula (I) does exhibit surface activity at pH 6 in phosphate buffer. The maximum surface pressure change is 11 mN/m and the critical micelle concentration is determined to be 0.1 mg/mL. Therefore, surface activity may support stabilization of super-saturation of this drug at a specific pH range.

### Example 22

### PVP Tablets

Composition of Tablets without PVP

| | | Quantity |
|---|---|---|
| | | (mg)/ |
| Composition | % | Tab |
| Internal Phase | | |
| ELINOGREL-ALB.001 | 35.52 | 160.89 |
| Magnesium Oxide | 11.92 | 54.00 |
| Destab Calcium Carbonate 90S | 29.8 | 135.00 |
| Ultra | | |
| PVPP XL | 4.857 | 22.00 |
| Magnesium Stearate | 0.883 | 4.00 |
| Aerosil 200PH | 0.883 | 4 |
| Poloxalkol | 7.285 | 33 |
| Cellulose MK GR | 5.102 | 23.11 |
| Magnesium Steatate | 0.883 | 4 |
| Basic lack White | 0.517 | 2.34 |
| Basic lack Yellow | 1.693 | 7.67 |
| Basic lack Red | 0.517 | 2.34 |
| Basic lack Black | 0.143 | 0.65 |
| Total | | 453 |

Composition of Tablets with PVP

| | | Quantity |
|---|---|---|
| | | (mg)/ |
| | | Tab |
| Composition | % | 150 mg |
| ELINOGREL-ALB.001 | 37.05 | 175.61 |
| PVP K30 | 4.219 | 20.00 |
| Magnesium Oxide | 11.39 | 54.00 |
| Destab Calcium Carbonate 90S Ultra | 28.48 | 135.00 |
| PVPP XL | 4.641 | 22.00 |
| Poloxalkol | 6.962 | 33 |
| Cellulose MK GR | 2.614 | 12.39 |
| Aerosil 200PH | 0.844 | 4 |
| Magnesium Steatate | 0.844 | 4 |
| Core total | | 460 |
| Basic lack White | 0.532 | 2.52 |
| Basic lack Yellow | 1.743 | 8.26 |
| Basic lack Red | 0.532 | 2.52 |
| Basic lack Black | 0.148 | 0.7 |
| Total | 100 | 474 |

### Dissolution Profile Tablets

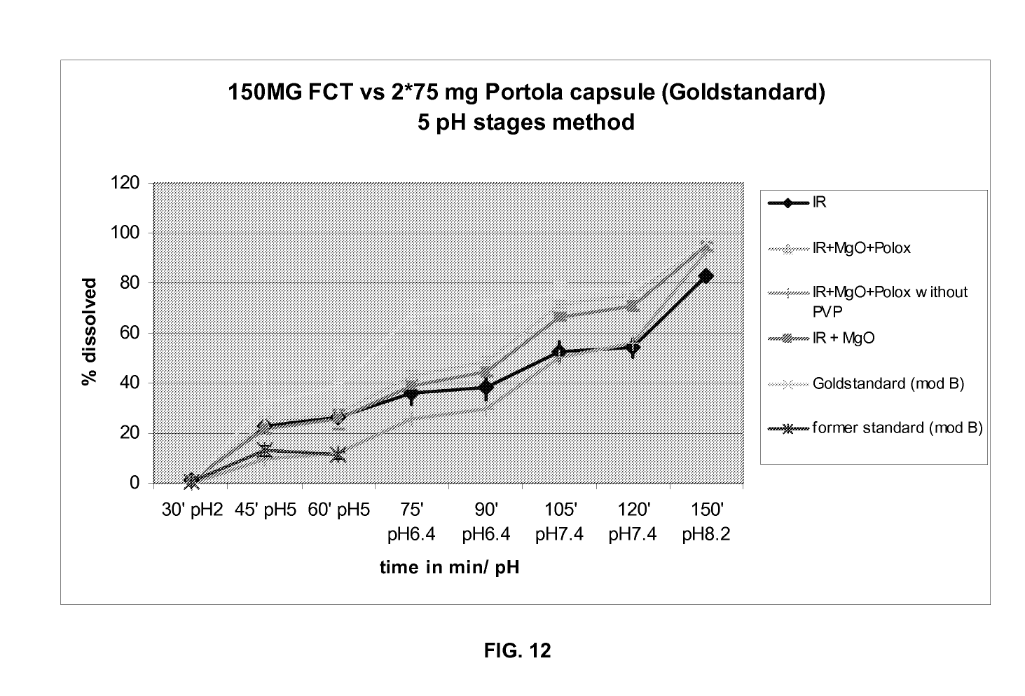

Tablets with PVP are denoted in the graph as IR+MgO+Polox (see FIG. 12). Tablets without PVP are denoted in the graph as IR+MgO+Polox without PVP. A significantly higher dissolution profile can be observed when using PVP.

In addition the Elinogrel displays a surface tension lowering effect in the pH range of 6.0 to 7.5. At pH of 9.5 no surface tension lowering in water could be observed. The CMC of the surface tension was determined at pH 6.0 with about 0.1 mg/ml and at pH 6.5 with about 1 mg/ml. The super-saturation could be stabilized by micelle forming as observed at pH 6.0-7.5. The highest stabilizing effect is given for the lowest CMC, in this case, 0.1 mg/ml at pH 6.0. Therefore, a buffer system that is able to maintain pH 6.0 for a longer period of time even after dilution will stabilize in addition the super-saturated state of the drug.

Dosage forms that may use of this system include solid dosage forms with PVP in the core of the tablet using or not using buffer systems, referenced herein that have a sufficient buffer capacity at pH 6.0. Suitable buffer systems are systems comprising solid pharmaceutical excipients, e.g. citric acid/Na₂HPO₄ di-hydrate, Na citrate/citric acid and the like, that could be used in solid dosage forms.

The disclosure of the following patent documents are herein incorporated by reference in their entirety for all purposes: U.S. Patent Application Publication Nos. US/2007/0123547; US/2009/0042916; US/2009/0156620; US/2007/0208045; and PCT Patent Application Publication Nos. WO2007/056219; WO2008/137809; WO2008/1137753, WO2008/1174809, WO2010/054020; and WO2007/056167.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A solid pharmaceutical composition comprising: a) at least about 15% by weight elinogrel or a pharmaceutically acceptable salt thereof relative to the total weight of the overall pharmaceutical composition, and b) at least one pharmaceutically acceptable carrier.

2. The solid pharmaceutical composition of claim 1 comprising: a) at least about 20% by weight elinogrel or a pharmaceutically acceptable salt thereof relative to the total weight of the overall pharmaceutical composition, and b) at least one pharmaceutically acceptable carrier.

3. The solid pharmaceutical composition of claim 1 comprising: a) about 15% to about 90% by weight elinogrel or a pharmaceutically acceptable salt thereof relative to the total weight of the overall pharmaceutical composition, and b) at least one pharmaceutically acceptable carrier.

4. The solid pharmaceutical composition of claim 1 comprising: a) about 20% to about 40% by weight elinogrel or a pharmaceutically acceptable salt thereof relative to the total weight of the overall pharmaceutical composition, and b) at least one pharmaceutically acceptable carrier.

5. The solid pharmaceutical formulation of claim 1 wherein the carrier is selected from the group consisting of: an alkalizer, a disintegrant, a crystallization inhibitor, a solubilizer, a filler, a polymer, a glidant and a lubricant.

6. (canceled)

7. (canceled)

8. The solid pharmaceutical formulation of claim 5 wherein the alkalizer is selected from the group consisting of magnesium oxide, calcium carbonate, calcium phosphate and combinations thereof.

9.-14. (canceled)

15. The solid pharmaceutical formulation of claim 5 wherein the disintegrant is selected from the group consisting of croscarmellose, sodium starch glycolate and crospovidone.

16.-22. (canceled)

23. The solid pharmaceutical formulation of claim 5 wherein the crystallization inhibitor is selected from the group consisting of a polyvinylpyrrolidone) and a hydroxypropylmethylcellulose.

24.-30. (canceled)

31. The composition of claim 1 wherein the elinogrel is substantially in form A.

32. (canceled)

33. The composition according to claim 1 which is in the form of a tablet.

34.-35. (canceled)

36. A compressed solid oral dosage form comprising: a) solid pharmaceutical composition comprising: a) at least about 15% elinogrel or a pharmaceutically acceptable salt thereof by weight relative to the total weight of the overall pharmaceutical composition, and b) at least one pharmaceutically acceptable carrier.

37. The compressed solid dosage form according to claim 36 wherein the active agent comprises elinogrel or a pharmaceutically acceptable salt thereof in a unit dosage of from between about 50 and 2000 mg.

38. The compressed solid dosage form according to claim 36 wherein the active agent comprises elinogrel or a pharmaceutically acceptable salt thereof in a unit dosage of from between about 100 and 1000 mg.

39.-43. (canceled)

44. The composition according to claim 36 comprising an additional therapeutic agent.

45. A method of treating or preventing a thrombotic condition in a mammal in need thereof, which method comprises administering to said mammal an effective amount of a solid pharmaceutical composition comprising: a) at least about 15% by weight elinogrel or a pharmaceutically acceptable salt thereof relative to the total weight of the overall pharmaceutical composition, and b) at least one pharmaceutically acceptable carrier.

46. The method of treating or preventing a thrombotic condition in a mammal in need thereof of claim 45, which method comprises administering to said mammal an effective amount of a solid pharmaceutical composition comprising: a) at least about 20% by weight elinogrel or a pharmaceutically acceptable salt thereof relative to the total weight of the overall pharmaceutical composition, and b) at least one pharmaceutically acceptable carrier.

47. The method of claim 46 wherein the thrombotic condition is chronic coronary heart disease or acute coronary syndrome.

48. A method to aid in dissolving elinogrel, comprising the step of: providing elinogrel in a composition with an alkalizer selected from the group consisting of calcium carbonate, magnesium oxide and calcium phosphate; a disintegrant selected from the group consisting of croscarmellose sodium, sodium starch glycolate and crospovidone; and optionally a crystallization inhibitor selected from the group consisting of a polyvinylpyrrolidone) and a hydroxypropylmethylcellulose in an amount of from at least about 3% by weight relative to the total weight of the overall pharmaceutical composition.

49. A method of producing a solid pharmaceutical composition comprising elinogrel; by contacting a) at least about 15% elinogrel or a pharmaceutically acceptable salt thereof by weight relative to the total weight of the overall pharmaceutical composition, with b) at least one pharmaceutically acceptable carrier.

50. The method of producing a solid pharmaceutical composition comprising elinogrel of claim 50; by contacting a) at least about 20% elinogrel or a pharmaceutically acceptable salt thereof by weight relative to the total weight of the overall pharmaceutical composition, with b) at least one pharmaceutically acceptable carrier.
